SEQUENCE LISTING (41 sequences)

SEQ ID NO: 1 - DNA, 28 bp, Artificial Sequence
  Description of Artificial Sequence Synthetic consensus sequence
  modified_base (1)..(5): a, c, t, g, unknown or other
  modified_base (8)..(8): a, c, t, g, unknown or other
  modified_base (19)..(19): a, c, t, g, unknown or other
  modified_base (22)..(28): a, c, t, g, unknown or other
nnnnnccncgcgcgcgcgnggnnnnnnn

SEQ ID NO: 2 - DNA, 14 bp, Unknown
  Description of Unknown Target sequence
  modified_base (3)..(3): a, c, t, g, unknown or other
  modified_base (12)..(12): a, c, t, g, unknown or other
ccncgcgcgcgngg

SEQ ID NO: 3 - DNA, 27 bp, Escherichia coli
gtcagttcgcgcacacacaggatgtcg

SEQ ID NO: 4 - DNA, 28 bp, Escherichia coli
gaggaaggcgcgcgcgtactgtgcggcg

SEQ ID NO: 5 - DNA, 28 bp, Escherichia coli
atttatcgtctacgggcaggggaagtgc

SEQ ID NO: 6 - DNA, 28 bp, Escherichia coli
atttatcgtctacgggcagggcaagtgc

SEQ ID NO: 7 - DNA, 28 bp, Escherichia coli
tgttgtcccctgcgctcgcgcaacgaaa

SEQ ID NO: 8 - DNA, 28 bp, Escherichia coli
atcaccgcctgcgcgcagccactctgcc

SEQ ID NO: 9 - DNA, 28 bp, Escherichia coli
atcaccgccagcgcgcagccactcttgc

SEQ ID NO: 10 - DNA, 28 bp, Escherichia coli
gcttggtccgctggtgcgcggtttaccg

SEQ ID NO: 11 - DNA, 28 bp, Escherichia coli
tgatatcccgtgcacgcgcggattaagc

SEQ ID NO: 12 - DNA, 28 bp, Escherichia coli
ctttttcccgcaggcgcgaggcgaagcc

SEQ ID NO: 13 - DNA, 28 bp, Escherichia coli
atcagaccgtttcccgcgtggtgaacca

SEQ ID NO: 14 - DNA, 33 bp, Escherichia coli
gcatgtggacgacgtcatcccactgatggcaga

SEQ ID NO: 15 - DNA, 33 bp, Escherichia coli
ggcagtgcgcccttatccgcatcagctggaaga

SEQ ID NO: 16 - DNA, 35 bp, Artificial Sequence
  Description of Artificial Sequence Synthetic primer
aagcccaatttactactcgttctggtgtttctcgt

SEQ ID NO: 17 - DNA, 35 bp, Artificial Sequence
  Description of Artificial Sequence Synthetic primer
acgagaaacaccagaacgagtagtaaattgggctt

SEQ ID NO: 18 - DNA, 45 bp, Artificial Sequence
  Description of Artificial Sequence Synthetic oligonucleotide
ctacacgacgctcttccgatctgcccaatttactactcgttctgg

SEQ ID NO: 19 - DNA, 28 bp, Artificial Sequence
  Description of Artificial Sequence Synthetic oligonucleotide
gtgttccccgcgccagcggggataaacc

SEQ ID NO: 20 - DNA, 28 bp, Artificial Sequence
  Description of Artificial Sequence Synthetic oligonucleotide
gtgttccccgcgcgcgcggggataaacc

SEQ ID NO: 21 - DNA, 149 bp, Artificial Sequence
  Description of Artificial Sequence Synthetic polynucleotide
aaggttggtgggttgtttttatgggaaaaaatgctttaagaacaaatgtatacttttagagagttccccgcgccagcggggataaaccgcccaatttactactcgttctggtgtttctcgtgagttccccgcgccagcggggataaacc

SEQ ID NO: 22 - DNA, 149 bp, Artificial Sequence
  Description of Artificial Sequence Synthetic polynucleotide
ggcgcaggcggggctggcgatcccggcgttaggcaccacgcacgctgactacttctttggtgatatcccgtgcacgcgcggattaagcgcccaatttactactcgttctggtgtttctcgttgatatcccgtgcacgcgcggattaagc

SEQ ID NO: 23 - DNA, 149 bp, Artificial Sequence
  Description of Artificial Sequence Synthetic polynucleotide
cagttgccccgcccagtcgaaaatatcctgatacagctggcgatggatagtgcgcaaatgcggtaaaccgcgcaccagcggaccaagcgcccaatttactactcgttctggtgtttctcgtcggtaaaccgcgcaccagcggaccaagc

SEQ ID NO: 24 - DNA, 149 bp, Artificial Sequence
  Description of Artificial Sequence Synthetic polynucleotide
tttccagacgggcgataaagccaggtagtttgttaaagacttcggcgctccagtgcggccctttttcccgcaggcgcgaggcgaagccgcccaatttactactcgttctggtgtttctcgtctttttcccgcaggcgcgaggcgaagcc

SEQ ID NO: 25 - RNA, 28 nt, Escherichia coli
guguuccgcccgccagggcggauaaacc

SEQ ID NO: 26 - RNA, 29 nt, Escherichia coli
ugaguaucccgugcacgcgcggauuaagc

SEQ ID NO: 27 - RNA, 28 nt, Escherichia coli
gcuugguccgcccggccgcgguuuaccg

SEQ ID NO: 28 - RNA, 29 nt, Escherichia coli
cuuuuuucccgcaggcgcgaggcgaagcc

SEQ ID NO: 29 - DNA, 27 bp, Artificial Sequence
  Description of Artificial Sequence Synthetic consensus sequence
  modified_base (7)..(9): a, c, t, g, unknown or other
  modified_base (12)..(14): a, c, t, g, unknown or other
  modified_base (21)..(24): a, c, t, g, unknown or other
aaaaaannnttnnnwatcaannnnttr

SEQ ID NO: 30 - DNA, 27 bp, Escherichia coli
aaaaaatcattaattaataataggtta

SEQ ID NO: 31 - DNA, 28 bp, Escherichia coli
cagatctccgcatacgcgcgggtacgcg

SEQ ID NO: 32 - DNA, 28 bp, Escherichia coli
gtaccgcccgcgcctgcgctgaaagagg

SEQ ID NO: 33 - DNA, 28 bp, Escherichia coli
aatttcccctcacccgtgcggatttagc

SEQ ID NO: 34 - DNA, 28 bp, Escherichia coli
gcagtcacccgccgcgcgcggatgcacc

SEQ ID NO: 35 - DNA, 28 bp, Escherichia coli
cgctatccctcgcccgcaggcgaaaacc

SEQ ID NO: 36 - DNA, 28 bp, Escherichia coli
tattttcctgcgcctgcgcggcagcttc

SEQ ID NO: 37 - DNA, 24 bp, Sulfolobus islandicus
gctaatctactatagaattgaaag

SEQ ID NO: 38 - DNA, 24 bp, Sulfolobus islandicus
gttatagattaaatagctagaaac

SEQ ID NO: 39 - DNA, 24 bp, Sulfolobus islandicus
gtaacaacacaaagaaactaaaac

SEQ ID NO: 40 - DNA, 37 bp, Sulfolobus islandicus
tcccgttggggttcaaaaacgtgagtataaaagaatt

SEQ ID NO: 41 - DNA, 37 bp, Sulfolobus islandicus
tccccctcgggctcaaaaacgtgaatatgaaagaact

(19) United States
(12) Patent Application Publication
(10) Pub. No.: US 2020/0190534 A1
(43) Pub. Date: Jun. 18, 2020

(54) Method of Off-Target Recording of Spacer Sequences within a Cell In Vivo

(71) Applicant: President and Fellows of Harvard College, Cambridge, MA (US)

(72) Inventors: Jeffrey Matthew Nivala, Allston, MA (US); Seth Lawler Shipman, Boston, MA (US); George M. Church, Brookline, MA (US)

(21) Appl. No.: 16/608,226

(22) PCT Filed: Apr. 27, 2018
(86) PCT No.: PCT/US18/29893

§ 371 Date: Oct. 25, 2019

Related U.S. Application Data
(60) Provisional application No. 62/490,901, filed on Apr. 27, 2017.

(51) Int. Cl. C12N 15/85; C12N 9/22; C12N 15/11

(52) U.S. Cl. CPC C12N 15/85; C12N 9/22; C12N 2800/107; C12N 2800/80; C12N 2310/20; C12N 15/111

(57) ABSTRACT

This invention provides methods of altering a cell including providing the cell with a nucleic acid sequence encoding a Cas1 protein and/or a Cas2 protein of a CRISPR adaptation system, providing the cell with a consensus CRISPR array nucleic acid sequence including a leader sequence and at least one consensus repeat sequence, and wherein the cell expresses the Cas1 protein and/or the Cas2 protein.

### RELATED APPLICATION DATA

This application claims priority to U.S. Provisional Application No. 62/490,901 filed on Apr. 27, 2017, which is hereby incorporated herein by reference in its entirety for all purposes.

### STATEMENT OF GOVERNMENT INTERESTS

This invention was made with government support under Grant Nos. 4R01MH103910-04 and 5R01MH103910-04 awarded by National Institutes of Mental Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 27, 2018, is named 010498_01086_WO_SL.txt and is 8,742 bytes in size.

### BACKGROUND

DNA is unmatched in its potential to encode, preserve, and propagate information (G. M. Church, Y. Gao, S. Kosuri, Next-generation digital information storage in DNA. Science 337, 1628 (2012); published online EpubSep 28 (10.1126/science.1226355)). The precipitous drop in DNA sequencing cost has now made it practical to read out this information at scale (J. Shendure. H. Ji. Next-generation DNA sequencing. Nat Biotechnol 26, 1135-1145 (2008); published online EpubOct (10.1038/nbt1486)). However, the ability to write arbitrary information into DNA, in particular within the genomes of living cells, has been restrained by a lack of biologically compatible recording systems that can exploit anything close to the full encoding capacity of nucleic acid space.

A number of approaches aimed at recording information within cells have been explored (D. R. Burrill, P. A. Silver, Making cellular memories. Cell 140, 13-18 (2010); published online EpubJan 8 (10.1016/j.cell.2009.12.034)). These systems can be broadly divided into those that encode events at the transcriptional level using feedback loops and toggles (N. T. Ingolia. A. W. Murray. Positive-feedback loops as a flexible biological module. Current biology: CB 17, 668-677 (2007); published online EpubApr 17 (10.1016/j.cub.2007.03.016), C. M. Ajo-Franklin, D. A. Drubin, J. A. Eskin, E. P. Gee, D. Landgraf, 1. Phillips, P. A. Silver, Rational design of memory in eukaryotic cells. Genes & development 21, 2271-2276 (2007); published online EpubSep 15 (10.1101/gad.1586107), D. R. Burrill, M. C. Inniss, P. M. Boyle, P. A. Silver, Synthetic memory circuits for tracking human cell fate. Genes & development 26, 1486-1497 (2012); published online EpubJul 1 (10.1101/gad.189035.112), T. S. Gardner, C. R. Cantor, J. J. Collins. Construction of a genetic toggle switch in Escherichia coli. Nature 403, 339-342 (2000); published online EpubJan 20 (10.1038/35002131). D. Greber, M. D. El-Baba, M. Fussenegger, Intronically encoded siRNAs improve dynamic range of mammalian gene regulation systems and toggle switch. Nucleic acids research 36, el01 (2008); published online EpubSep (10.1093/nar/gkn443), M. R. Atkinson. M. A. Savageau, J. T. Myers, A. J. Ninfa, Development of genetic circuitry exhibiting toggle switch or oscillatory behavior in Escherichia coli. Cell 113, 597-607 (2003); published online EpubMay 30, H. Kobayashi, M. Kaern, M. Araki, K. Chung, T. S. Gardner, C. R. Cantor, J. J. Collins, Programmable cells: interfacing natural and engineered gene networks. Proc Natl Acad Sci USA 101, 8414-8419 (2004); published online EpubJun 1 (10.1073/pnas.0402940101), N. Vilaboa. M. Fenna, J. Munson, S. M. Roberts, R. Voellmy, Novel gene switches for targeted and timed expression of proteins of interest. Molecular therapy: the journal of the American Society of Gene Therapy 12, 290-298 (2005); published online EpubAug (10.1016/j.ymthe.2005.03.029), B. P. Kramer, M. Fussenegger, Hysteresis in a synthetic mammalian gene network. Proc Nat Acad Sci USA 102, 9517-9522 (2005); published online EpubJul 5 (10.1073/pnas.0500345102), D. R. Burrill, P. A. Silver, Synthetic circuit identifies subpopulations with sustained memory of DNA damage. Genes & development 25, 434-439 (2011); published online EpubMar 1 (10.1101/gad.1994911). M. Wu. R. Q. Su, X. Li, T. Ellis, Y. C. Lai, X. Wang, Engineering of regulated stochastic cell fate determination. Proc Natl Acad Sci USA 110, 10610-10615 (2013); published online EpubJun 25 (10.1073/pnas.1305423110)), versus those that encode information permanently into the genome, most often employing recombinases to store information via the orientation of DNA segments (T. S. Ham, S. K. Lee, J. D. Keasling, A. P. Arkin, Design and construction of a double inversion recombination switch for heritable sequential genetic memory. PLoS One 3, e2815 (2008)10.1371/journal.pone.0002815). T. S. Moon, E. J. Clarke. E. S. Groban, A. Tamsir, R. M. Clark, M. Eames, T. Kortemme, C. A. Voigt, Construction of a genetic multiplexer to toggle between chemosensory pathways in Escherichia coli. Journal of molecular biology 406, 215-227 (2011); published online EpubFeb 18 (10.1016/j.jmb.2010.12.019), J. Bonnet, P. Subsoontorn. D. Endy, Rewritable digital data storage in live cells via engineered control of recombination directionality. Proc Natl Acad Sci USA 109, 8884-8889 (2012); published online EpubJun 5 (10.1073/pnas.1202344109), L. Yang, A. A. Nielsen, J. Fernandez-Rodriguez, C. J. McClune. M. T. Laub, T. K. Lu, C. A. Voigt, Permanent genetic memory with >1-byte capacity. Nat Methods 11, 1261-1266 (2014); published online EpubDec (10.1038/nmeth.3147), P. Siuti, J. Yazbek. T. K. Lu, Synthetic circuits integrating logic and memory in living cells. Nat Biotechnol 31, 448-452 (2013); published online EpubMay (10.1038/nbt.2510)). While the majority of these systems are effectively binary, more recent efforts have also been made toward analogue recording systems (F. Farzadfard, T. K. Lu. Synthetic biology. Genomically encoded analog memory with precise in vivo DNA writing in living cell populations. Science 346. 1256272 (2014); published online EpubNov 14 (10.1126/science.1256272)) and digital counters (A. E. Friedland, T. K. Lu. X. Wang, D. Shi, G. Church, J. J. Collins, Synthetic gene networks that count. Science 324, 1199-1202 (2009); published online EpubMay 29 (10.1126/science.172005)). Despite these efforts, the recording and genetic storage of little more than a single byte of information (L. Yang. A. A. Nielsen, J. Fernandez-Rodriguez, C. J. McClune, M. T. Laub, T. K. Lu. C. A. Voigt, Permanent genetic memory with >1-byte capacity. Nat Methods 11, 1261-1266 (2014); published online EpubDec (10.1038/nmeth.3147)) has remained out of reach.

Immunological memory is essential to an organism's adaptive immune response, and hence must be an efficient and robust form of recording molecular events into living cells. The CRISPR-Cas system is a recently understood form of adaptive immunity used by prokaryotes and archaea (R. Barrangou, C. Fremaux, H. Deveau, M. Richards, P. Boyaval, S. Moineau, D. A. Romero. P. Horvath, CRISPR provides acquired resistance against viruses in prokaryotes. Science 315, 1709-1712 (2007); published online EpubMar 23 (10.1126/science.1138140)). This system remembers past infections by storing short sequences of viral DNA within a genomic array. These acquired sequences are referred to as protospacers in their native viral context, and spacers once they are inserted into the CRISPR array. Importantly, new spacers are integrated into the CRISPR array ahead of older spacers (I. Yosef. M. G. Goren, U. Qimron, Proteins and DNA elements essential for the CRISPR adaptation process in Escherichia coli. Nucleic acids research 40, 5569-5576 (2012); published online EpubJul (10.1093/nar/gks216)). Over time, a long record of spacer sequences can be stored in the genomic array, arranged in the order in which they were acquired. Thus, the CRISPR array functions as a high capacity temporal memory bank of invading nucleic acids. However, there is a need for a CRISPR-Cas system that can direct recording of specific and arbitrary DNA sequences into the genome of prokaryotic and eukaryotic cells.

### SUMMARY

The present disclosure provides materials and methods where DNA protospacer sequences within a genetically modified cell can be introduced and recorded as spacer sequences into a noncanonical CRISPR array within the genome of the cell or within a plasmid within the cell using an integration complex, such as a bacterial integration complex as is known in the art, such as a Cas1-Cas2 integration complex. The noncanonical CRISPR array (as distinguished from a canonical CRISPR array) is an off-target location or integration site for spacer acquisition which may be referred to herein as a “neo-CRISPR array.” The repeat sequence of a “neo-CRISPR array” may be homologous to the repeat sequence of a canonical CRISPR array. The sequence of the “neo-CRISPR arrays” can be determined to create a consensus sequence which may be used to function as a repeat sequence in a CRISPR array. Such a CRISPR array including a consensus repeat sequence may be referred to as a “consensus CRISPR array.” According to aspects described herein, a consensus CRISPR array includes a repeat sequence which is a consensus sequence of a plurality of repeat sequences located within off-target integration sites or noncanonical integration sites. According to aspects described herein, a consensus CRISPR array includes a leader sequence which may be a consensus sequence of a plurality of leader sequences located within off-target integration sites or noncanonical integration sites. According to one aspect, a consensus CRISPR array includes a repeat sequence which is a consensus sequence of a plurality of repeat sequences located within off-target integration sites or noncanonical integration sites and a leader sequence which may be a consensus sequence of a plurality of leader sequences located within off-target integration sites or noncanonical integration sites.

According to one aspect, methods are provided for identifying a plurality of off-target spacer integration sites within a cell, such as E. coli. According to one aspect, the plurality of off-target integration sites are used to generate a consensus repeat sequence for the plurality of off-target integration sites, such that the integration factor or complex can recognize and use the consensus repeat sequence to integrate a spacer sequence into the nucleic acid sequence including the consensus repeat sequence. The consensus repeat sequence is included within a cell, optionally along with a leader sequence, which forms a consensus CRISPR array and is used as an integration site for one or more or a plurality of protospacer sequences using an integration complex, such as a Cas1-Cas2 integration complex. It is to be understood that one or skill will readily identify integration factors or complexes, such as bacterial integration complexes, and their corresponding canonical CRISPR array leader and repeat sequences. One aspect of the present disclosure is to identify off target integration sites for a particular species of integration complex, and then determine a consensus sequence for either the leader sequence or repeat sequence or both to create a consensus CRISPR array sequence and then to incorporate the consensus CRISPR array sequence into a cell for use in integrating spacer sequences therein. In this manner, spacer integration may be more efficient using a consensus CRISPR array sequence compared to a canonical CRISPR array sequence for a given integration factor or complex.

According to methods described herein, the one or more or a plurality of protospacer sequences can be generated by the cell or within the cell or may be provided as species exogenous to the cell or may be introduced into the cell from outside the cell. Once inserted into the consensus CRISPR array, the spacer sequence can be used to create a functional guide RNA, such as for genome editing purposes.

According to one aspect, a method of altering a cell is provided. The method includes providing the cell with one or more nucleic acid sequences encoding an integration factor or factors which alone or together form an integration complex, such as an Cas1 protein and/or a Cas2 protein of a CRISPR adaptation system, providing the cell with a consensus CRISPR array nucleic acid sequence including a leader sequence and at least one repeat sequence which is a consensus sequence of a plurality of repeat sequences within off-target integration sites, wherein the cell expresses the integration factor or factors, such as the Cas1 protein and/or the Cas2 protein, and wherein the consensus CRISPR array nucleic acid sequence is within genomic DNA of the cell or on a plasmid. According to one aspect, the nucleic acid sequence encoding the integration factor or factors, such as the Cas1 protein and/or a Cas2 protein, is provided to the cell within a vector or within one or more vectors.

According to one aspect, methods described herein include providing the cell with a protospacer sequence which may be a natural DNA sequence or a synthetic DNA sequence, whether defined or undefined, known or unknown. According to one aspect, the protospacer sequence includes a modified “AAG” protospacer adjacent motif (PAM). According to one aspect, the protospacer is endogenous or exogenous. According to one aspect, the protospacer is provided to the cell as an exogenous nucleic acid sequence using methods known to those of skill in the art. According to one aspect, the cell is altered by inserting the protospacer sequence into the consensus CRISPR array nucleic acid sequence to form an inserted spacer sequence.

In certain embodiments, the cell is a prokaryotic or a eukaryotic cell. In one embodiment, the prokaryotic cell is E. coli. In another embodiment, the E. coli is BL21-AI. In one embodiment, the eukaryotic cell is a yeast cell, plant cell or a mammalian cell. In certain embodiments, the cell lacks endogenous Cas1 and Cas2 proteins. In one embodiment, the nucleic acid sequence encoding the Cas1 protein and/or a Cas2 protein includes one or more inducible promoters for induction of expression of the Cas1 and/or Cas2 protein. In another embodiment, the nucleic acid sequence encoding the Cas1 protein and/or a Cas2 protein includes a first regulatory element operable in a eukaryotic cell. In one embodiment, the nucleic acid sequence encoding the Cas1 protein and/or a Cas2 protein is codon optimized for expression of Cas1 and/or Cas2 in a eukaryotic cell.

According to another aspect, an engineered, non-naturally occurring cell is provided. In one embodiment, the cell includes one or more nucleic acid sequences encoding a Cas1 protein and/or a Cas2 protein of a CRISPR adaptation system wherein the cell expresses the Cas1 protein and/or the Cas2 protein. In another embodiment, the cell includes a consensus CRISPR array nucleic acid sequence including a leader sequence and at least one consensus repeat sequence, wherein the consensus CRISPR array nucleic acid sequence is inserted within genomic DNA of the cell or on a plasmid. According to one aspect, the cell is provided with a protospacer sequence to be introduced into the consensus CRISPR array as an inserted spacer sequence.

According to one aspect, an engineered, non-naturally occurring cell is provided. In one embodiment, the cell includes one or more nucleic acid sequences encoding a Cas1 protein and/or a Cas2 protein of a CRISPR adaptation system, and a consensus CRISPR array nucleic acid sequence including a leader sequence and at least one consensus repeat sequence, wherein the cell expresses the Cas1 protein and/or the Cas2 protein, and wherein the CRISPR array nucleic acid sequence is inserted within genomic DNA of the cell or on a plasmid.

According to another aspect, a method of inserting a target DNA sequence within genomic DNA of a cell is provided. In one embodiment, the method includes providing the cell with target DNA sequence and wherein the cell includes one or more nucleic acid sequences encoding a Cas1 protein and/or a Cas2 protein of a CRISPR adaptation system and a consensus CRISPR array nucleic acid sequence including a leader sequence and at least one consensus repeat sequence, wherein the cell expresses the Cas1 protein and/or the Cas2 protein and wherein the consensus CRISPR array nucleic acid sequence is within genomic DNA of the cell or on a plasmid, and wherein the target DNA sequence is under conditions within the cell wherein the Cas1 protein and/or the Cas2 protein processes the target DNA and the target DNA is inserted into the consensus CRISPR array nucleic acid sequence adjacent a corresponding consensus repeat sequence. In one embodiment, the target DNA sequence is a protospacer as described herein. In another embodiment, the target DNA protospacer is a defined synthetic DNA or a naturally occurring endogenous DNA. In yet another embodiment, the target DNA sequence includes a modified “AAG” protospacer adjacent motif (PAM). In certain embodiments, a plurality of target DNA sequences are provided to the cell and are inserted into the consensus CRISPR array nucleic acid sequence at corresponding consensus repeat sequences. In one embodiment, the one or more nucleic acid sequences encoding the Cas1 protein and/or a Cas2 protein is provided to the cell within a vector.

According to one aspect, a nucleic acid storage system is provided. In one embodiment, the nucleic acid storage system includes an engineered, non-naturally occurring cell including one or more nucleic acid sequences encoding a Cas1 protein and/or a Cas2 protein of a CRISPR adaptation system, a consensus CRISPR array nucleic acid sequence including a leader sequence and at least one consensus repeat sequence, wherein the cell expresses the Cas1 protein and/or the Cas2 protein and wherein the cell is provided as described herein with one or more or a plurality of protospacer DNA sequences, wherein the consensus CRISPR array nucleic acid sequence is within genomic DNA of the cell or on a plasmid, and wherein the one or more nucleic acid sequences encoding a Cas1 protein and/or a Cas2 protein is within genomic DNA of the cell or on one or more plasmids. In one embodiment, at least one oligonucleotide sequence within the cell includes protospacer that is processed and inserted as a spacer sequence into the consensus CRISPR array nucleic acid sequence.

According to another aspect, a method of recording molecular events into a cell is provided. In one embodiment, the method includes generating or providing a DNA sequence or sequences containing information about the molecular events in the cell wherein the cell includes one or more nucleic acid sequences encoding a Cas1 protein and/or a Cas2 protein of a CRISPR adaptation system and a consensus CRISPR array nucleic acid sequence including a leader sequence and at least one consensus repeat sequence, wherein the cell expresses the Cas1 protein and/or the Cas2 protein and wherein the consensus CRISPR array nucleic acid sequence is within genomic DNA of the cell or on a plasmid, wherein the one or more nucleic acids encoding the Cas1 protein and/or the Cas2 protein is within genomic DNA of the cell or on a plasmid, and wherein the DNA sequence is generated or provided under conditions within the cell wherein the Cas1 protein and/or the Cas2 protein processes the DNA and the DNA is inserted into the consensus CRISPR array nucleic acid sequence adjacent a corresponding consensus repeat sequence. In certain embodiments, the step of generating or providing is repeated such that a plurality of DNA sequences is inserted into the consensus CRISPR array nucleic acid sequence at corresponding consensus repeat sequences. In one embodiment, the DNA sequence includes a protospacer. In yet another embodiment, the protospacer is a defined synthetic DNA. In one embodiment, the DNA sequence includes a modified “AAG” protospacer adjacent motif (PAM). In certain embodiments, the molecular events comprise transcriptional dynamics, molecular interactions, signaling pathways, receptor modulation, calcium concentration, and electrical activity. In one embodiment, the recorded molecular events are decoded. In another embodiment, the decoding is by sequencing. In yet another embodiment, the decoding by sequencing comprises using the order information from pairs of acquired spacers in single cells to extrapolate and infer the order information of all recorded sequences within the entire population of cells. In one embodiment, the plurality of DNA sequences is recorded into a specific genomic locus of the cell in a temporal manner. In another embodiment, the DNA sequence is recorded into the genome of the cell in a sequence and/or orientation specific manner.

According to another aspect, a system for in vivo molecular recording is provided. In one embodiment, the system includes an engineered, non-naturally occurring cell including one or more nucleic acid sequences encoding a cas1 protein and/or a cas2 protein of a CRISPR adaptation system, and a consensus CRISPR array nucleic acid sequence including a leader sequence and at least one consensus repeat sequence, wherein the cell expresses the Cas1 protein and/or the Cas2 protein and wherein the consensus CRISPR array nucleic acid sequence is within genomic DNA of the cell or on a plasmid.

According to one aspect, the disclosure provides a kit of directed recording of molecular events into a cell comprising an engineered, non-naturally occurring cell including a nucleic acid sequence encoding a Cas1 protein and/or a Cas2 protein of a CRISPR adaptation system, and a consensus CRISPR array nucleic acid sequence including a leader sequence and at least one consensus repeat sequence, wherein the cell expresses the Cas1 protein and/or the Cas2 protein and wherein the consensus CRISPR array nucleic acid sequence is within genomic DNA of the cell or on a plasmid.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 is schematic directed to the genesis of a neo-CRISPR array. FIG. 1 discloses SEQ ID NO: 2.

FIGS. 2A-2C are directed to whole-genome deep sequencing methods that identify off-target spacer integration events within the E. coli genome. FIG. 2A is a schematic of experimental workflow. A culture of E. coli BL21 expressing Cas1 and Cas2 is electroporated with a 35 bp oligo protospacer that includes a 5′ AAG PAM. Following electroporation and outgrowth, the total DNA content of the cells is isolated, fragmented, and shotgun sequenced on an Illumina high-throughput sequencing machine. Reads are mapped back to the BL21 reference genome. Spacer integration events are identified as an about 61 bp insertion, that includes the spacer sequence (33 bp) and the duplicated target site (about 28 bp repeat). FIG. 2B is a schematic of the E. coli, genome and integration sites. Eight of the off-target integration sites discovered within the genome are shown in the diagram labeled as the gene in which they were inserted. Integrations of the oligo protospacer are shown in red, while the blue lines denote the integration of genome-derived spacers. The origin of the dashed arrows indicates the site of the genome-derived spacer and point toward the site of integration. Off-target integration events within the lac1 gene are not shown because they cannot be unambiguously mapped to the genome or plasmid. FIG. 2C is a graph comparing the number of on-target integrations into the first position of the CRISPR1 array and off-target integrations elsewhere in the genome outside of the CRISPR1 array. The protospacer source is denoted in red or blue for oligo or genome-derived spacers, respectively.

FIG. 3 is a table listing off-target spacer integrations identified by whole-genome sequencing. Genomic integration site nucleotide numbering and gene annotations referenced to E. coli BL21 genome GenBank accession number CP010816. FIG. 3 discloses the “repeat 1” sequences as SEQ ID NOS 3-5, 7-8 and 10-13, the “repeat 2” sequences as SEQ ID NOS 3-4, 6-7 and 9-13, and the “spacer” sequences as SEQ ID NOS 14-15, all respectively, in order of appearance.

FIG. 4 is a representation of a Weblogo of the nine off-target integration sites identified by whole-genome sequencing, aligned to the BL21 CRISPR1 array leader and repeat sequence.

FIG. 5 is a table of nucleotide sequences used in the Examples. psAA33 (for/rev): forward and reverse oligo strands of the protospacer used for defined spacer acquisition. MiSeq_M13F: forward primer used for specific amplification of genomic fragments containing psAA33 integrations. Repeat sequences used in plasmid-based array spacer acquisition experiment: Native repeat and off-target consensus repeat (mutations in red). M13-KI2/NCA^{araD/fsc/hsdR}: synthetic arrays cloned into the pJKR plasmid used in the primed-acquisition assays. Bold: leader, Italics: repeats. Underlined: M13 spacer. FIG. 5 discloses SEQ ID NOS 16-24, respectively, in order of appearance.

FIGS. 6A-6F are directed to a method (Spacer-seq) used to identify hundreds of off-target spacer integration sites within the E. coli genome. FIG. 6A is a schematic of Spacer-seq workflow. FIG. 6B depicts a genome diagram showing an example of a single Spacer-seq experiment with the number of reads mapped to the E. coli BL21 genome (binned per 10 kb). Dashed lines represent 100 reads. FIG. 6C is a graph of percent of Spacer-seq reads mapped to a CRISPR array, or to off-target sites within the genome or expression plasmid. Error bars represent mean±SD, n=4 biological replicates. FIG. 6D is a graph comparing the average number of off-target integration events mapped to the genome or plasmid, normalized by total DNA content within the cell (assuming about 30 plasmids/cell). Error bars represent mean±SD, n=4 biological replicates. FIG. 6E is a representation of a Weblogo of the about 700 unique off-target integration sites identified by Spacer-seq, aligned to the BL21 CRISPR1 array leader and repeat sequence. FIG. 6F is a graph of percent of expanded arrays after defined spacer acquisition experiment. Plasmid containing the minimal version of the K12 CRISPR1 array (native repeat) is compared to a mutant version with repeat mutations C14G and A15C (off-target consensus repeat). Error bars represent mean±SEM. n=3 biological replicates. * denotes p<0.05 calculated with a two-sample unpaired t-test.

FIGS. 7A-7B depicts off-target sites identified by Spacer-Seq. FIG. 7A depicts genome diagrams showing 4 Spacer-seq biological replicates, mapped to the E. coli BL21 genome. Unique integration sites per 10 kb, the dashed lines represent 1 site. FIG. 7B is a plasmid diagram mapping all the unique off-target integrations sites identified by Spacer-seq reads generated from 4 biological replicates, mapped to the pWUR_1+2 plasmid. Note that the lacI gene has been removed from the map because reads mapping to lac1 cannot be unambiguously mapped to the genome of plasmid.

FIG. 8 is a table of off-target integration sites within the BL21 genome discovered by Spacer-seq. The table lists the genomic site of integration, whether it was forward or reverse strand, and the number of reads/counts for each unique site. R1-R4 are separate biological replicates. Sites within the lacI gene (which cannot be unambiguously mapped between the genome or plasmid) are denoted.

FIGS. 9A-9D are directed to a comparison of three different neo-CRISPR array sequences and their activity in primed acquisition. FIG. 9A is a schematic of the plasmid-based neo-CRISPR arrays used in the primed acquisition assays. The arrays contain an inducible promoter driving expression of 60 nt of the off-target leader (leader^{NCA}, cyan) along with a 33 nt spacer matching the M13 phage genome (spacer^{M13}, red) that is flanked by the 28 nt off-target repeat sequences (repeat^{NCA}, yellow). FIG. 9B depict multiple sequence alignment of the neo-CRISPR array repeats aligned to the BL21 CRISPR repeat sequence. Residues conserved with the BL21 repeat are shown in black. FIG. 9B discloses SEQ ID NOS 19, 11, 10 and 12, respectively, in order of appearance. FIG. 9C is a graph of results of the primed-acquisition assay with the strains harboring the plasmids encoding either wildtype (BL21) or NCA (araD, fic, and hsdR) arrays containing the M13 spacer, or a strain with no plasmid-based array (-plas). Error bars represent mean±SD. n=3 biological replicates. ** denotes p<0.01 calculated with a two-sample unpaired t-test. FIG. 9D depicts an RNA secondary structure comparison (as predicted by Mfold) of the BL21 CRISPR and neo-CRISPR repeat sequences. The free energy (AG) of each structure is also shown. FIG. 9D discloses SEQ ID NOS 25-28, respectively, in order of appearance.

FIGS. 10A-10G depict that Spacer-seq identifies hundreds of off-target spacer integration sites within the E. coli genome. FIG. 10A depicts a schematic of Spacer-seq workflow. (i) Fragmentation of isolated genomic DNA containing defined spacer acquisition events. (ii) Ligation of adaptor sequences onto fragment ends. (ii) PCR amplification using the defined spacer sequence and adaptor sequence as primers for (iv) specific enrichment of fragments containing spacer insertions. (iv) High-throughput sequencing of enriched fragments and mapping of reads to reference genome. FIG. 10B depicts a genome diagram that shows an example of a single Spacer-seq experiment with the number of reads mapped to the E. coli BL21 genome (binned per 10 kb). Dashed lines represent 100 reads. FIG. 10C shows percent of Spacer-seq reads mapped to a CRISPR array, or to off-target sites within the genome or expression plasmid. Error bars represent mean±SD, n=3 biological replicates. FIG. 10D shows a comparison between the average number of off-target integration events mapped to the genome or plasmid, normalized by total DNA content within the cell (assuming ˜30 plasmids/cell). Error bars represent mean±SD. n=3 biological replicates. FIG. 10E depicts a weblogo of the ˜700 unique off-target integration sites identified by Spacer-seq. aligned to the BL21 CRISPR1 array leader and repeat sequence. FIG. 10F depicts percent of expanded arrays after defined spacer acquisition experiment. Plasmid containing the minimal version of the K12 CRISPR1 array (native repeat) is compared to a mutant version with repeat mutations C14G and A15C (OTCR). Error bars represent mean±SEM. n=3 biological replicates. * denotes p=0.04 calculated with a two-sample unpaired t-test. FIG. 10G depicts percent of expanded arrays after defined spacer acquisition experiment. The genomic CRISPR1 array (native repeat) is compared to a strain in which the entire CRISPR1 locus is replaced with a minimal array consisting of a 100 nt leader and a single mutant repeat (OTCR). Error bars represent mean±SEM. n=3 biological replicates. ** denotes p=0.002 calculated with a two-sample unpaired t-test.

FIGS. 11A-11B depict spacer integration efficiency and off-target frequency under varying induction conditions. FIG. 11A. Black bars and red bars denote the percentage of oligo-expanded arrays and percentage of off-target spacer-seq reads following DSA, respectively. Experiments were repeated (n=3) with different relative levels of Cas1-Cas2 induction. 1×, 0.1× and 0× correspond to 1 mM IPTG+0.02% arabinose, 0.1 mM IPTG+0.002% arabinose, and no inducers added, respectively. FIG. 11B. Same as in FIG. 11A, but with different concentrations of supplied oligo protospacers. 1×, 0.1× and 0× correspond to 3.2 uM, 0.32 uM, and 0.032 uM oligos, respectively. n=3 biological replicates. Error bars represent mean±SD for all panels.

FIGS. 12A-12D depict effects of genomic knockouts of IHF and the CRISPR1 locus on off-target spacer integration activity. FIG. 12A depicts percentage of oligo integrations into the CRISPR1 locus (gray) or off-target site (red) normalized per cell (array) following DSA in the BL21-AI strain (WT), or the BL21-AI strain with either the IHF-alpha (ΔIHFα) or IHF-beta (ΔIHFβ) subunits knocked out. Error bars represent mean±SD. FIG. 12B depicts percentage of spacer-seq reads aligned on-target to the CRISPR1 locus (CRISPR1 gray) or to other regions in the genome (off-target, red) in the WT, ΔIHFα, and ΔIHFβ strains. Error bars represent mean±SD. FIG. 12C depicts pearson correlation coefficient (R) of the off-target site identities between the WT vs ΔIHFα/β strain, WT vs ΔCRISPR1 strain, and WT vs WT replicates. Error bars represent mean±SD. FIG. 12D depicts percentage of spacer-seq reads aligned to unique off-target sites within the genome. Knockout strain percentages (y-axis) of the ΔIHFα/strains (cyan) and ΔCRISPR1 strain (red) are compared to those of WT (x-axis). Each point represents a unique genomic site. For all panels, n=4 (for WT) and n=3 (for knockout strains) biological replicates.

FIG. 13 depicts potential IHF binding sites located near the top 10 most frequent off-target integration sites. The structure of the native CRISPR1 array is shown at the top. The leader has a segment (cyan) that shares 93% sequence homology to the IHF consensus binding site sequence. The top 10 most frequent off-target sites across spacer-seq data sets are shown below. The arrows and numbers denote integration site location within the BL21 genome. The red regions signify the duplicated repeat sequences. Cyan shows regions within 100 bp up- and downstream of the repeat that have the highest homology to the IHF binding site consensus sequence. Exact percent sequence identity is shown above each segment. FIG. 13 discloses SEQ ID NOS 29-30, respectively, in order of appearance.

FIG. 14 depicts effect of plasmid-based CRISPR arrays on off-target spacer integration frequency within IHF knockout strains. Integrations into the plasmid-based array are most frequently on target, and decrease the overall fraction of off-target integrations into the genome within the IHF knock strains (ΔIHFα and ΔIHFβ). n=3 biological replicates. Error bars mean±SD.

FIGS. 15A-15B depict transcription of off-target spacer integration products. FIG. 15A depicts comparison of the frequency of off-target spacer-seq reads derived from spacer-seq performed on whole genome (DNA) or whole transcriptome (RNA) isolated samples following DSA. Reads mapping to ribosomal operons (cyan) are enriched within the RNA spacer-seq data sets. FIG. 15B depicts that overall, RNA spacer-seq reads (red) are enriched for highly transcribed regions of the genome, compared to DNA Spacer-seq reads (black).

FIGS. 16A-16E depict comparison of three different neo-CRISPR array sequences and their activity in target interference and primed acquisition. FIG. 16A depicts schematic of the plasmid-based neo-CRISPR arrays used in the primed acquisition assays. The arrays contain an inducible promoter driving expression of 60 nt of the off-target leader (leader^{NCA}, cyan) along with a 33 nt spacer matching the M13 phage genome (spacer^{M13}, red) that is flanked by the 28 nt off-target repeat sequences (repeat^{NCA}, yellow). FIG. 16B depicts multiple sequence alignment of the neo-CRISPR array repeats aligned to the BL21 CRISPR repeat sequence. Residues conserved with the BL21 repeat are shown in black. FIG. 16B discloses SEQ ID NOS 19, 11, 31, 10, 12 and 32-36, respectively, in order of appearance. FIG. 16C depicts results of the plasmid interference assay with the strains harboring the plasmids encoding either wildtype (BL21) or NCA arrays containing the M13 spacer, or a strain with no plasmid-based array. n=4 biological replicates. Error bars represent mean±SD. FIG. 16D depict results of the primed-acquisition assay with the strains harboring the plasmids encoding either wildtype (BL21) or NCA arrays containing strains. n=3 biological replicates. Error bars represent mean±SD. FIG. 16E depicts comparison of plasmid-based NCA expansion frequencies following DSA. Expansion frequencies for each NCA were quantified by high-throughput sequencing of the plasmid-based arrays. Each point represents the percent of expansions detected for each array. We did not detect any expansions for NCAs that do not display a point (ie. fic, potG, and yfic), indicating integration efficiencies below 10⁻⁴ percent.

FIGS. 17A-17F depict evidence for native off-target spacer integrations. FIG. 17A depicts a diagram of Y. pestis phylogeny and presence or absence of CRISPR arrays YPb and YPc, as denoted by a green check or red X, respectively. The dashed line demarks the branch between the absence/presence of the YPb and YPc arrays along the lineage. Figure adapted from [27]. FIG. 17B depicts that Y. pestis contains three canonical CRISPR arrays (YPa, YPb, and YPc) and one set of type I-F Cas genes. Each array within the CO92 genome contains a leader (L), sharing 63% sequence identity across all three 200 nt leaders, and between 3-8 spacers (S) separated by 100% identical repeat sequences (R) with the exception of the terminal repeats, which are degenerate (D). The Y. pestis Angola strain, which is considered to be an ancestral strain of the species, contains only the Cas-proximal array (YPa). At the Angola genome locations homologous to the C092 arrays. YPb and YPc, there are hypothetical protein coding regions (hyp. prot.) that only contain the corresponding YPb and YPc array leader and terminal/degenerate repeat sequence. The putative spacer integration off-target site (between the pre-neo-CRISPR leader (green) and pre-neo-CRISPR “degenerate” repeat (red)) within the ancestral Angola genome that eventually generated the YPb and YPc arrays of the descendant C092 strain are demarcated. Gray regions within the dashed lines have 100% sequence homology. FIG. 17C depicts a diagram of S. islandicus phylogeny and presence or absence of putative an off-target integration site within the genome at 1,813,802 (numbering based on M.16.4 genome), as denoted by a green check or red X, respectively. The REY15A strain does not have a complete second repeat site. Figure adapted from [29]. FIG. 17D depicts a diagram comparing genomic features of S. islandicus strains M*. L*, and Y* with those of the LAL14/1 and HVE10/3 strains at the location of a putative off-target spacer integration event within the latter strains. The repeat and spacer regions are highlight in red and yellow, respectively. FIG. 17E depicts the off-target repeat shares sequence homology with the other two canonical CRISPR repeat sequence types present within the species (the S. islandicus lineage contains three distinct CRISPR-Cas types: IA, IIIB-Cmr α, and IIIB-Cmr-β). FIG. 17E discloses SEQ ID NOS 37-39, respectively, in order of appearance. FIG. 17F depicts spacer sequence homology to a known S. islandicus plasmid pLD8501. FIG. 17F discloses SEQ ID NOS 40-41, respectively, in order of appearance.

FIG. 18 depicts BL21 CRISPR1 spacer expression levels as determined by RNA-Seq experiments. n=3 biological replicates, error bars denote mean±SD.

### DETAILED DESCRIPTION

Embodiments of the present disclosure are directed to methods of altering a cell via a CRISPR-Cas system. According to certain aspects, a bacterial integration factor or factors or complex known to those of skill in the art, such as the Cas1-Cas2 complex, integrates oligonucleotide spacers, whether synthetic or natural, into a consensus CRISPR array nucleic acid sequence that is within genomic DNA of the cell or on a plasmid. As protospacers, the oligonucleotide spacers may be produced within the cell or exogenously supplied to the cell from outside the cell and are processed and inserted into the consensus CRISPR array nucleic acid sequence as spacer sequences.

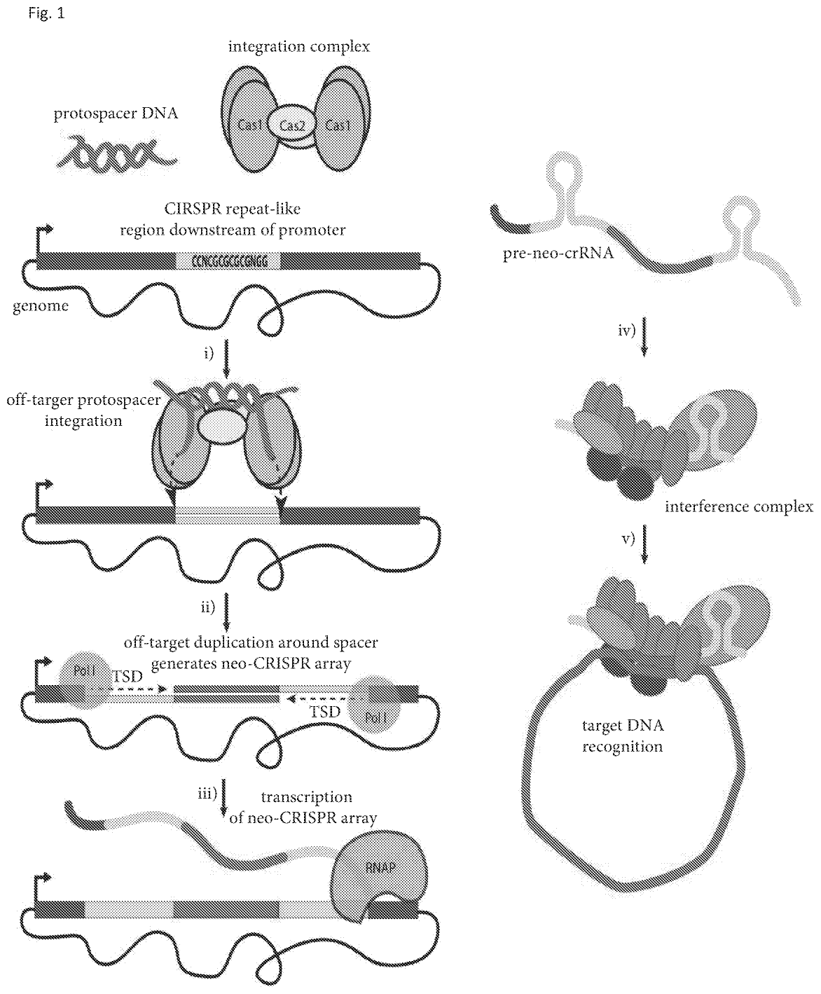

Aspects of the present disclosure are based on the discovery that off-target spacer integrations can occur at many unique off-target spacer integration sites throughout the E. coli genome and carried plasmids. The off-target integration sites are referred to herein as neo-CRISPR arrays. FIG. 1 describes the genesis of neo-CRISPR arrays: i) The Cas1-Cas2 integration complex captures a protospacer and binds a repeat-like sequence near a promoter at an off-target site within the genome; ii) protospacer integration and target site duplication generates neo-CRISPR array; iii) the neo-CRISPR array is transcribed into pre-neo-crRNA using nearby promoter activity: iv) Pre-neo-crRNA is processed into mature neo-crRNA and complexed with Cas interference proteins (e.g. Cascade): v) The neo-crRNA-interference complex targets complementary DNA.

As in canonical type I-E CRISPR acquisitions, these off-target integrations are accompanied by an about 28 nt target site duplication. A palindromic sequence motif closely matching the native CRISPR repeat sequence is also highly conserved within the off-target site repeats. Specific internal bases within repeat sequence facilitates recognition by the Cas1-Cas2 complex. The 60 nt upstream of the off-target sites (i.e. the off-target “leader” region) displays no conservation outside of the few bases proximal to the leader-repeat junction. According to one aspect, additional factors, such as IHF that binds a specific sequence in the native BL21 CRISPR1 leader and has been proposed as essential for in-vivo spacer acquisition¹³, are not required at a precise site within the leader for all integration events. Accordingly, aspects of the methods of the present disclosure may or may not include IHF, such as when off-target integration sites lack a strict IHF-binding site.

The terms “polynucleotide”, “nucleotide”, “nucleotide sequence”. “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

In general, “a CRISPR adaptation system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR adaption system is derived from a type I, type II, or type III CRISPR system. Cas and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type 11 systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In some embodiments, the disclosure provides for integration of defined synthetic DNA into a consensus CRISPR array in a directional manner, occurring preferentially, but not exclusively, adjacent to the leader sequence. In the type I-E system from E. coli, it was demonstrated that the first direct repeat, adjacent to the leader sequence is copied, with the newly acquired spacer inserted between the first and second direct repeats.

In one embodiment, the protospacer is an oligonucleotide sequence which may be a natural DNA sequence or a synthetic DNA sequence, whether defined or undefined. In some embodiments, the protospacer is at least 10, 20, 30, 40, or 50 nucleotides, or between 10-100, or between 20-90, or between 30-80, or between 40-70, or between 50-60, nucleotides in length. In one embodiment, the oligonucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei. Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., 120001, supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura. Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

### Bacterial Integration Factors or Complexes and Repeat Sequences

Exemplary integration factors are known to those of skill in the art and are available in the literature. Exemplary integration factors and/or complexes include Cas1 and Cas1 from E. coli and related prokaryotic CRISPR-Cas systems, the bacterial TrwC integrase, bacteriophage lambda integrase, MuA transposase, and HIV integrase.

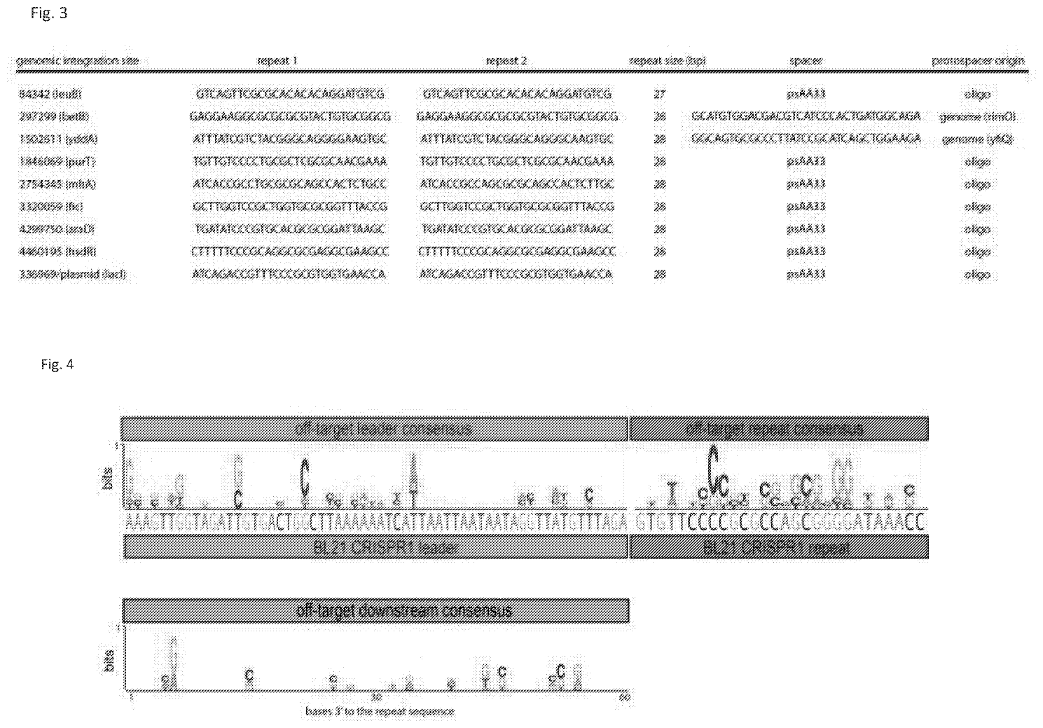

For a particular integration factor or complex, one of skill will readily be able to identify a canonical or naturally occurring corresponding CRISPR array sequence including a leader sequence and a repeat sequence. For example an exemplary canonical or naturally occurring CRISPR array leader and repeat sequence is depicted in FIG. 4 for BL21. The methods described herein include identifying a plurality of off-target integration sites and determining the sequence of the off-target integration sites. The sequences of the off-target integration sites are then analyzed to determine a consensus sequence. Methods of determining a consensus sequence from a plurality of sequences are described herein and are known to those of skill in the art and will be apparent based on the present disclosure. It is to be understood that the disclosure is not limited by the particular and exemplary off target integration sites determined in E. coli for the E. coli Cas1 and Cas2 integration complex, but that the methods described herein can be extended to other cells and integration factors or complexes. Further, it is to be understood that the disclosure is not limited by the particular and exemplary consensus repeat sequence described herein, but that the methods described herein can be extended to identify or generate or create other consensus repeat sequences depending on the particular integration factor or complex. An exemplary repeat consensus sequence created from off target integration sites is (5′)NNNNNCCNCGCGC GCGCGNGGNNNNNNN(3′) (SEQ ID NO: 1), wherein N is a nucleotide. An exemplary repeat consensus sequence created from off target integration sites (such as those shown in FIG. 3) is shown in FIG. 4.

### Target DNA Sequence

The term “target DNA sequence” includes a nucleic acid sequence which is to be inserted into a consensus CRISPR array nucleic acid sequence within the genomic DNA of the cell or on a plasmid according to methods described herein. The target DNA sequence may be referred to as a protospacer sequence or a spacer sequence. The target DNA sequence may be expressed by the cell or provided into the cell from outside the cell. According to one aspect, the target DNA sequence is naturally occurring within the cell. According to one aspect, the target DNA sequence is foreign to the cell (i.e., foreign nucleic acid sequence), such that it is not a naturally occurring sequence produced by the cell. According to one aspect, the target DNA sequence is non-naturally occurring within the cell. According to another aspect, the target DNA sequence is synthetic. According to one aspect, the target DNA has a defined sequence.

### Foreign Nucleic Acids

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources. According to one aspect, a foreign nucleic acid is exogenous to the cell. According to one aspect, a foreign nucleic acid is foreign, non-naturally occurring within the cell.

### Cells

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells. Particular cells include mammalian cells.

According to one aspect, the cell is a eukaryotic cell or a prokaryotic cell. According to one aspect, the cell is a yeast cell, bacterial cell, fungal cell, a plant cell or an animal cell. According to one aspect, the cell is a mammalian cell. According to one aspect, the cell is a human cell. According to one aspect, the cell is a stem cell whether adult or embryonic. According to one aspect, the cell is a pluripotent stem cell. According to one aspect, the cell is an induced pluripotent stem cell. According to one aspect, the cell is a human induced pluripotent stem cell. According to one aspect, the cell is in vitro, in vivo or ex vivo.

### Vectors

Vectors according to the present disclosure include those known in the art as being useful in delivering genetic material into a cell and would include regulators, promoters, nuclear localization signals (NLS), start codons, stop codons, a transgene etc., and any other genetic elements useful for integration and expression, as are known to those of skill in the art. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, bacteriophages, herpesviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector. “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and LipofectinM). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185. Academic Press, San Diego. Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol 11 promoters), one or more pol 1 promoters (e.g. 1, 2, 3, 4, 5, or more pol I promoters), or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags. FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### Example I

### Exogenous Protospacers are Inserted into Off-Target Sites in the Genome, as Well as into a Target CRISPR Array

The spacer acquisition process of the E. coli type I-E CRISPR-Cas system has been well characterized. See Wright. A. V., Nulez, J. K. & Doudna, J. A. Biology and applications of CRISPR systems: harnessing nature's txoolbox for genome engineering. Cell 164, 29-44 (2016) and Yosef, I., Goren, M. G. & Qimron, U. Proteins and DNA elements essential for the CRISPR adaptation process in Escherichia coli. Nucleic Acids Res. 40, 5569-5576 (2012) each of which is hereby incorporated by reference in its entirety. As is typical in all known types, spacer integration by this system requires two Cas proteins, Cas1 and Cas2, which form a heteromeric integration complex. See Yosef, I., Goren. M. G. & Qimron, U. Proteins and DNA elements essential for the CRISPR adaptation process in Escherichia coli. Nucleic Acids Res. 40, 5569-5576 (2012). K. Nuñez, A. S. Lee. A. Engelman. J. A. Doudna, Integrase-mediated spacer acquisition during CRISPR-Cas adaptive immunity. Nature 519, 193-198 (2015), and Nuñez, J. K. et al. Cas1-Cas2 complex formation mediates spacer acquisition during CRISPR-Cas adaptive immunity. Nat. Struct. Mol. Biol. 21, 528-534 (2014) each of which is hereby incorporated by reference in its entirety. On the protospacer, the Cas1-Cas2 complex recognizes a 3′ TTC 5′ protospacer adjacent motif (PAM) on the bottom strand that largely determines the efficiency and directionality of protospacer integration into the CRISPR array. See E. Savitskaya. E. Semenova, V. Dedkov. A. Metlitskaya, [symbol] K. Severinov, High-throughput analysis of type I-E CRISPR/Cas spacer acquisition in E. coli. RNA Biol. 10, 716-725 (2013), S. Shmakov et al., Pervasive generation of oppositely oriented spacers during CRISPR adaptation. Nucleic Acids Res. 42, 5907-5916 (2014). J. Wang et al., Structural and mechanistic basis of PAM-dependent spacer acquisition in CRISPR-Cas systems. Cell 163, 840-853 (2015), and Shipman, S. L., Nivala, J., Macklis, J. D., Church, G. M. Molecular recordings by directed CRISPR spacer acquisition. Science 353(6298). (2016) each of which is hereby incorporated by reference in its entirety. The array is minimally composed of 60 nt of the leader region, and a single 28 nt repeat. See Yosef, I., Goren, M. G. & Qimron, U. Proteins and DNA elements essential for the CRISPR adaptation process in Escherichia coli. Nucleic Acids Res. 40, 5569-5576 (2012) which is hereby incorporated by reference in its entirety. Within the array, Cas1-Cas2 recognizes a conserved inverted repeat (IR) motif within the interior of the repeat. A non-Cas protein, integration host factor (IHF), binds to a conserved sequence within the leader, and helps direct integration into the 5′ leader-proximal end of the array. See Nuñez, J. K., Bai, L., Harrington, L. B., Hinder, T. L. & Doudna. J. A. CRISPR immunological memory requires a host factor for specificity. Mol. Cell 62, 824-833 (2016) which is hereby incorporated by reference in its entirety.

According to certain aspects, in vivo specificity of off-target spacer integration at the whole genome scale was studied and off-target integration sites for spacer sequences within the E. coli genome were identified. The spacer sequences integrated within off-target integration sites may serve as functional crRNAs. The off target integration sites were studied to develop a consensus sequence which may serve as a repeat sequence in a CRISPR array nucleic acid sequence (termed a consensus CRISPR array) introduced into a cell. Methods are described as follows.

Defined Spacer Acquisition:

The process of defined spacer acquisition using electroporated oligos has been described. See Shipman, S. L., Nivala, J., Macklis, J. D., Church, G. M. Molecular recordings by directed CRISPR spacer acquisition. Science 353(6298). (2016) which is hereby incorporated by reference in its entirety. Briefly, liquid cultures of E. coli BL21-AI cells (Thermo) harboring a plasmid expressing Cas1 and Cas2 under the control of a T7-lac promoter (pWUR 1+2, which was a gift from Udi Qimron) were started from plates and grown overnight in LB. In the morning, cultures were diluted 1:30 into 3 mL fresh LB containing L-arabinose (Sigma-Aldrich) at a final concentration of 0.2% (w/w) and 1 mM isopropyl-beta-D-thiogalactopyranoside (IPTG; Sigma-Aldrich), and grown for an additional 2 hours. Cells were then pelleted, re-suspended, and washed in water three times to remove residual media. Cells were then re-suspended in 50 uL (per 1 mL of the 3 mL culture) of water containing the psAA33 forward and reverse oligo strands each at a concentration of 3.1 uM and electroporated with a Bio-Rad Gene Pulser set to 1.8 kV, 25 uF, and 200Ω. Immediately following electroporation, cells were re-suspended in fresh LB and allowed to recover overnight. In the morning, the cultures were pelleted and frozen at −20 until DNA extraction.

Whole-Genome Preparation, Sequencing, and Analysis:

The total DNA content of the cell pellets were extracted and purified with a QIAmp DNA Mini Kit (Qiagen) following the protocol for bacterial cultures. The isolated DNA was then sheared to ˜500 bp fragments using a Covaris S2 ultrasonicator. DNA fragments were then prepped for sequencing using NEBNext Ultra DNA Library Prep Kit for Illumina (NEB), and sequenced on an Illumina MiSeq machine (MiSeq reagent kit V2, paired-end 2×250 read lengths). Sequencing data was analyzed using the Geneious assembler (Biomatters) by aligning reads to the BL21 reference genome (GenBank accession number CP010816) allowing for up to 70 nt insertions, and manually searching for reads containing the psAA33 sequence or insertions of about 61 nt.

Spacer-Seq and Analysis:

Using the sheared and adaptor-ligated DNA fragments previously prepared for WGS as input, a PCR reaction was performed using a forward primer containing the NEBNext Adaptor sequence (5′) and a portion of the psAA33 sequence (3′) and a reverse primer matching the NEBNext Adaptor sequence. Enriched fragments were then indexed, and sequenced on an Illumina MiSeq machine. Sequencing data were then analyzed using custom written software (Python). Briefly, primer sequences were removed from the reads, and filtered for sequences that contained a match to the remaining psAA33 sequence not included in the primer. Sequences satisfying these criteria were then mapped to the BL21 reference genome.

Primed-Acquisition Assay and Analysis:

The Neo-CRISPR arrays containing the M13 spacer were synthesized as gBlocks (IDT) and cloned by Gibson assembly into the pJKR-H-tetR vector (replacing the GFP gene downstream of the pLtetO promoter). Sequence-verified plasmids were transformed into E. coli K12 BW40114. A primed-acquisition has been previously described. See Datsenko, K. A., Pougach, K., Tikhonov, A., Wanner, B. L., Severinov. K., and Semenova, E. Molecular memory of prior infections activates the CRISPR/Cas adaptive bacterial immunity system. Nat. Commun. 3, 945. (2012) which is hereby incorporated by reference in its entirety. Briefly, overnight cultures were started from plates. In the morning, cultures were diluted into fresh LB containing the inducers arabinose, IPTG, and anhydrotetracycline (aTc; Clontech), and grown for an additional two hours. Cells were then diluted 1:10 into fresh LB (with inducers) and M13KE phage (NEB) at a concentration of 1×10⁹ pfu/mL. Cultures were then grown overnight. In the morning, an aliquot of the sample was boiled and used as input for a PCR reaction that amplified the K12 CRISPR2 array locus. Amplicons were prepped with Illumina NEBNext Ultra DNA Library Prep Kit for Illumina (NEB), and sequenced on an Illumina MiSeq machine. Sequence data was analyzed using custom written software (Python). Briefly, the sequence of the first spacer within each array was extracted and blasted against a local database to quantify the number of spacers matching the M13 phage genome.

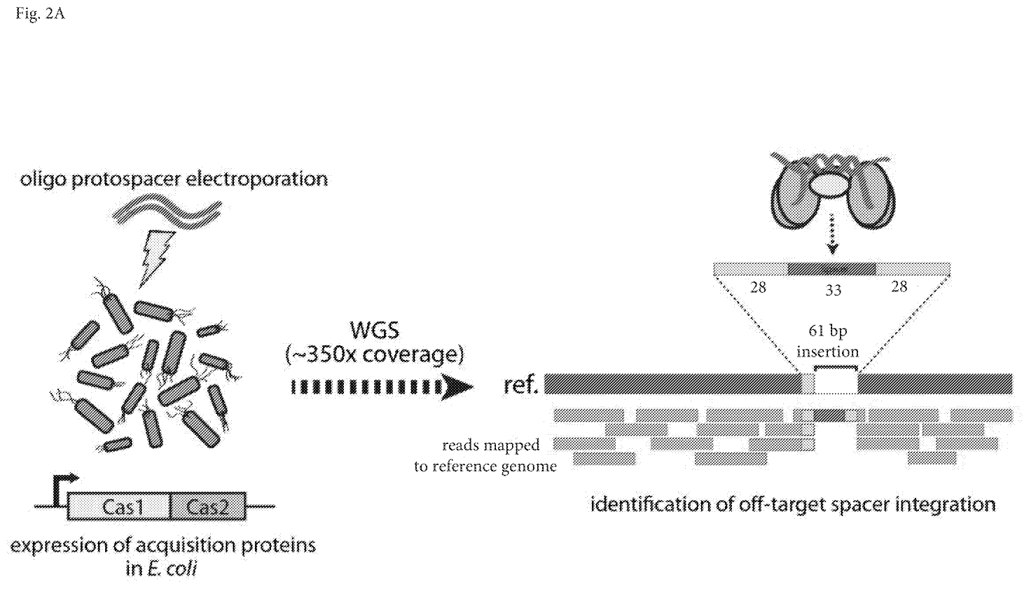

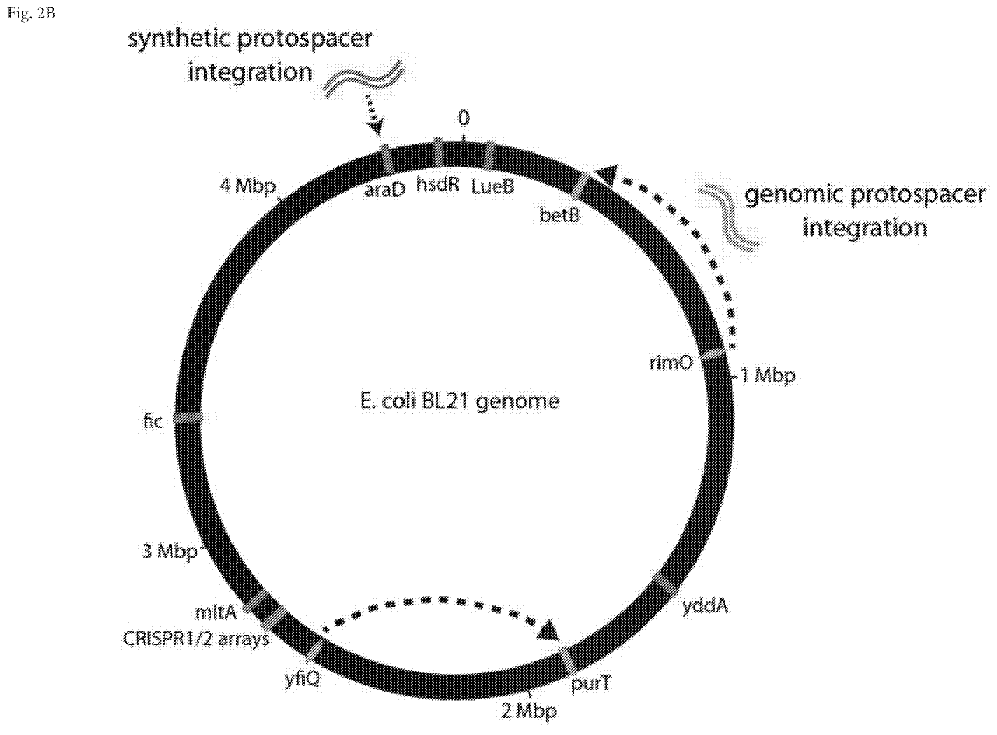

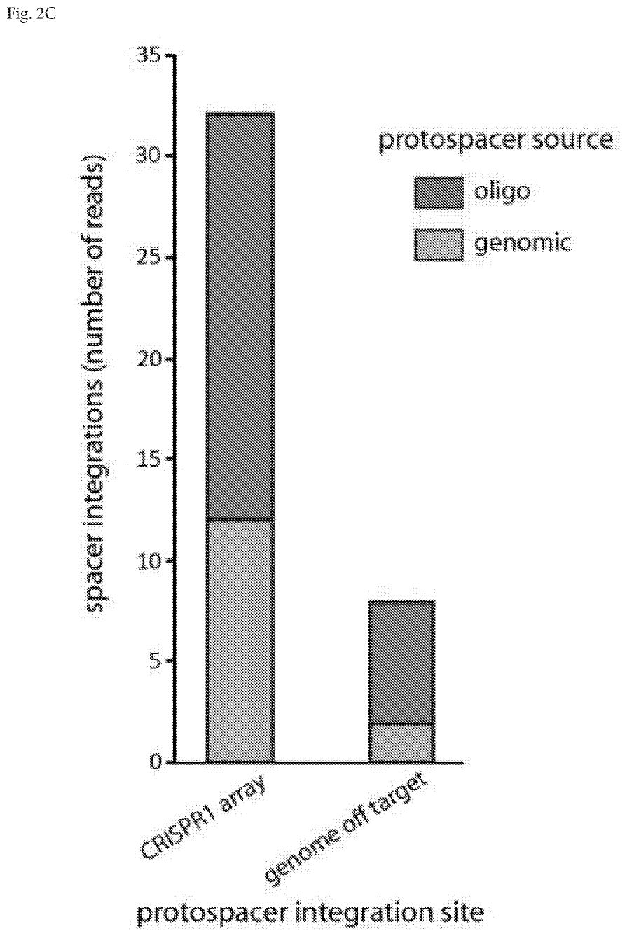

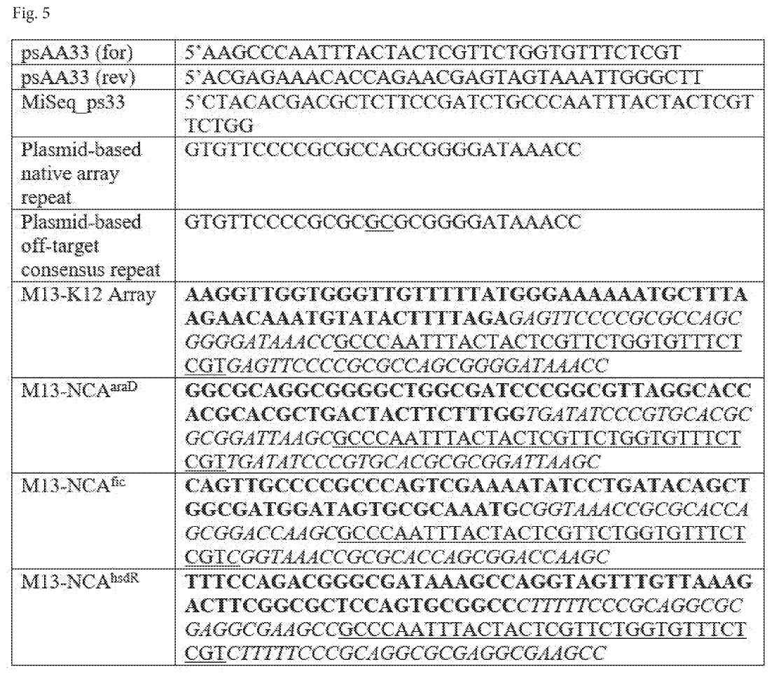

Electroporation of synthetic oligo protospacers into E. coli BL21 overexpressing Cas1-Cas2 is known to lead to acquisition of these oligo sequences into the genomic CRISPR1 locus. See Shipman. S. L., Nivala. J., Macklis, J. D., Church, G. M. Molecular recordings by directed CRISPR spacer acquisition. Science 353(6298) (2016) which is hereby incorporated in its entirety. A method referred to as defined spacer acquisition (DSA) was used to identify off-target spacer integrations within the genome where the spacer sequence is known a priori. Defined spacer acquisition was performed using a previously characterized oligo protospacer that is integrated with high efficiency (psAA33). See K. Nuñez, A. S. Lee, A. Engelman. J. A. Doudna, Integrase-mediated spacer acquisition during CRISPR-Cas adaptive immunity. Nature 519, 193-198 (2015) and Shipman, S. L., Nivala, J., Macklis, J. D., Church, G. M. Molecular recordings by directed CRISPR spacer acquisition. Science 353(6298)(2016) each of which is hereby incorporated by reference in its entirety. The psAA33 sequence matches a 35 nt segment of the M13 bacteriophage genome, and includes a canonical 5′ AAG PAM. Following electroporation of this oligo into cultures of cells overexpressing Cas1-Cas2, cells were diluted into fresh LB and allowed to recover. After culture outgrowth overnight, the total DNA content of the cells (genomic and plasmid) was extracted and subjected to whole-genome shotgun sequencing at a depth of about 350× genomic coverage on an Illumina MiSeq the results of which are shown in FIG. 2A. Reads were mapped to the BL21 reference genome. Analysis conditions were set to allow for alignments with greater than 50 nt insertions in each read (relative to the reference sequence), as canonical spacer integration into the CRISPR array results in a 61 nt expansion (33 nt spacer+28 nt repeat duplication). After mapping, 32 reads aligning to the first position of the genomic CRISPR1 array showed array expansions resulting from the integration of the psAA33 sequence (20 reads) or endogenous genome/plasmid-derived spacers (12 reads). A total of 9 reads were identified outside the genomic arrays (“off-target”) that similarly contained all the hallmarks of spacer integration events. Each of these reads contained a 27 or 28 nt region of the genome duplicated on both sides of a 33 nt insertion. In 7 instances, the 33 nt insertion contained the psAA33 sequence. In each of these, the inserted bases excluded the 5′ AA bases of the PAM as indicated in FIG. 2B and FIG. 3, which is consistent with Cas1-Cas2 mediated PAM processing and integration of the oligo spacer that occurs in “on-target” integrations. See J. Wang et al., Structural and mechanistic basis of PAM-dependent spacer acquisition in CRISPR-Cas systems. Cell 163, 840-853 (2015) and Shipman, S. L., Nivala. J., Macklis, J. D., Church. G. M. Molecular recordings by directed CRISPR spacer acquisition. Science 353(6298)(2016) each of which is hereby incorporated by reference in its entirety. The other 2 off-target instances were 33 nt spacer insertions whose sequences occur at other regions of the BL21 genome, and appear to be off-target integration events of genome-derived spacers. These results showed that, in these conditions, off-target integrations by Cas1-Cas2 occur with a frequency of about 1 off-target integration for every 4 on-target integrations into the CRISPR array, and that both oligo and genome-derived protospacers can be integrated off-target. See FIG. 2C. Overrepresented nucleotides were identified in the genomic sites surrounding the off-target integrations (See FIG. 4) that partially agreed with previous work characterizing essential array sequence motifs. See Wang, R., Ming, L., Gong, L., Hu, S., and Xiang, H. DNA motifs determining the accuracy of repeat duplication during CRISPR adaptation in Haloarcula hispanica. Nucleic Acids Res. 44(9):4266-77 (2016) and Moran G Goren, et al. Repeat Size Determination by Two Molecular Rulers in the Type I-E CRISPR Array. Cell Reports 16(11) (2016) each of which is hereby incorporated by reference in its entirety. FIG. 5 is a table of the nucleotides used in this Example.

### Example II

### Characterization of Many Off-Target Sites in the Genome Using Spacer-Seq

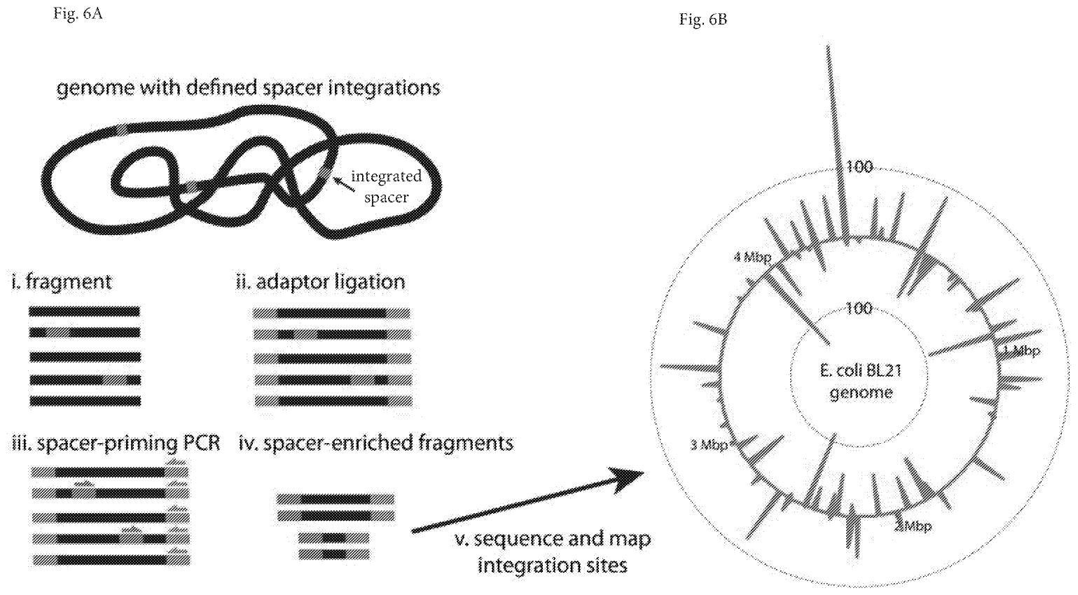

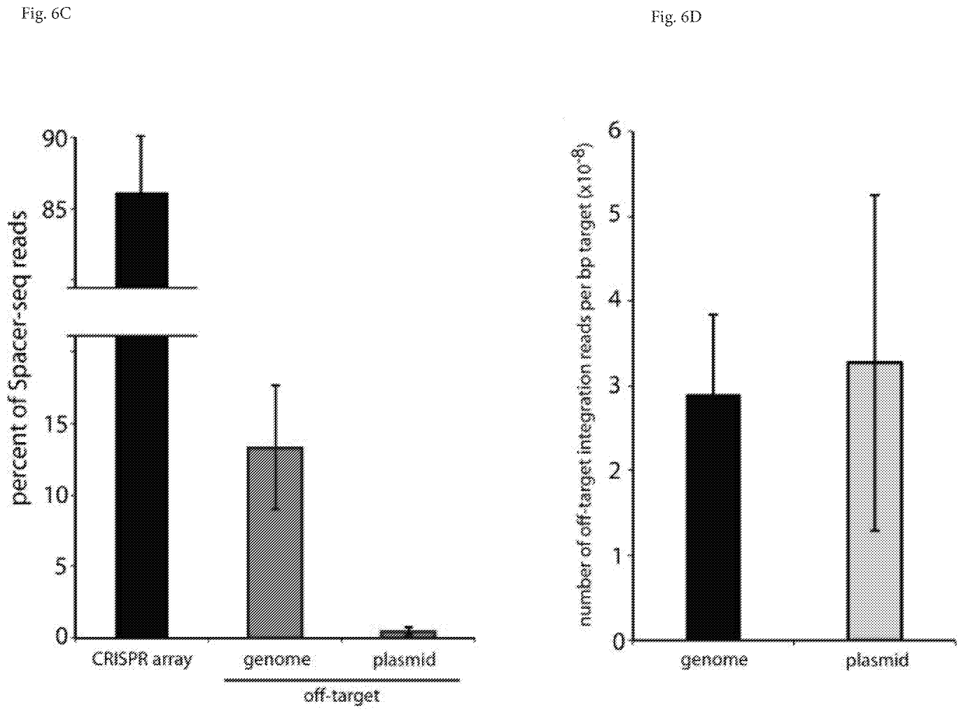

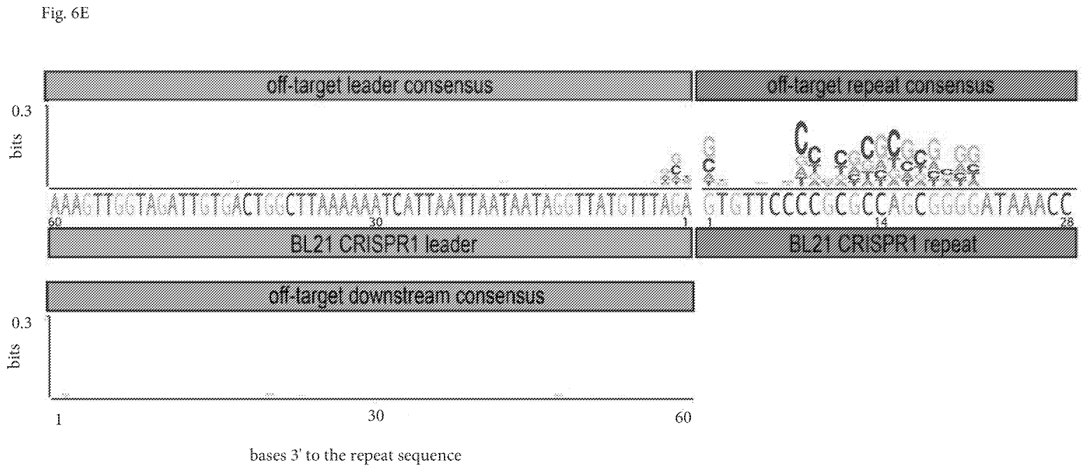

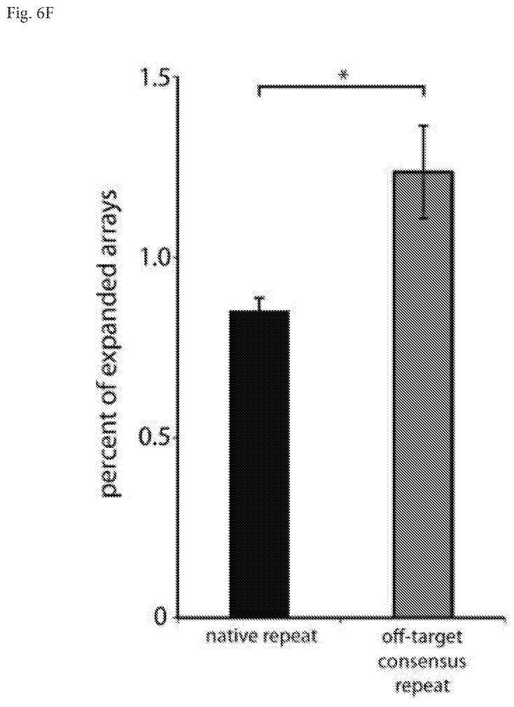

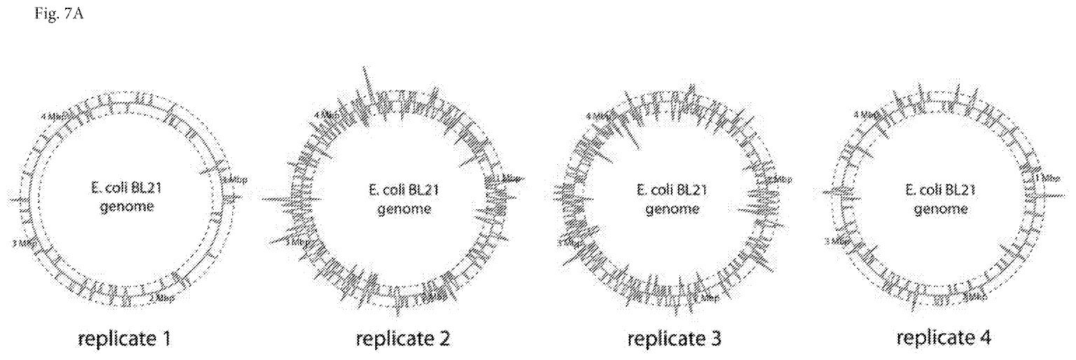

Off-target integration sites in addition to those identified in Example I were identified using a method termed “Spacer-seq” to target sequencing to spacer integration sites. The Spacer-seq method and results are shown in FIG. 6A-6F.

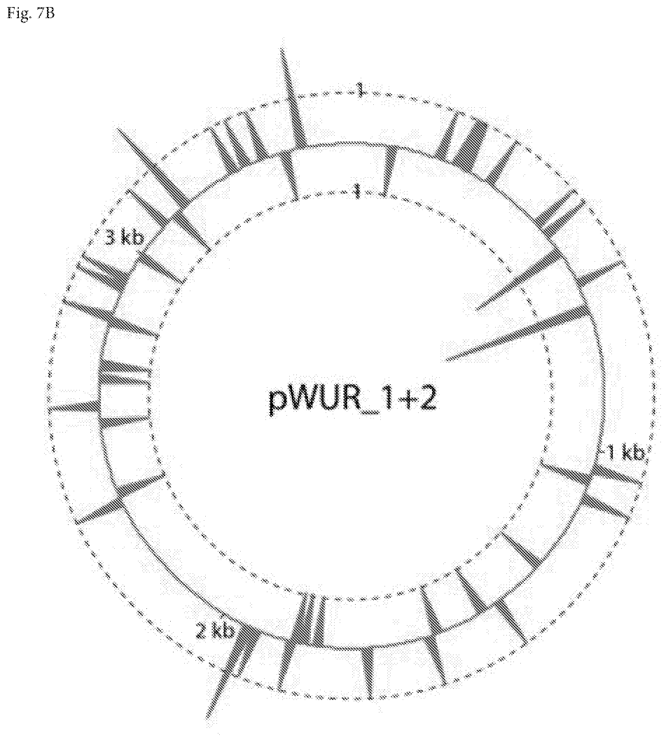

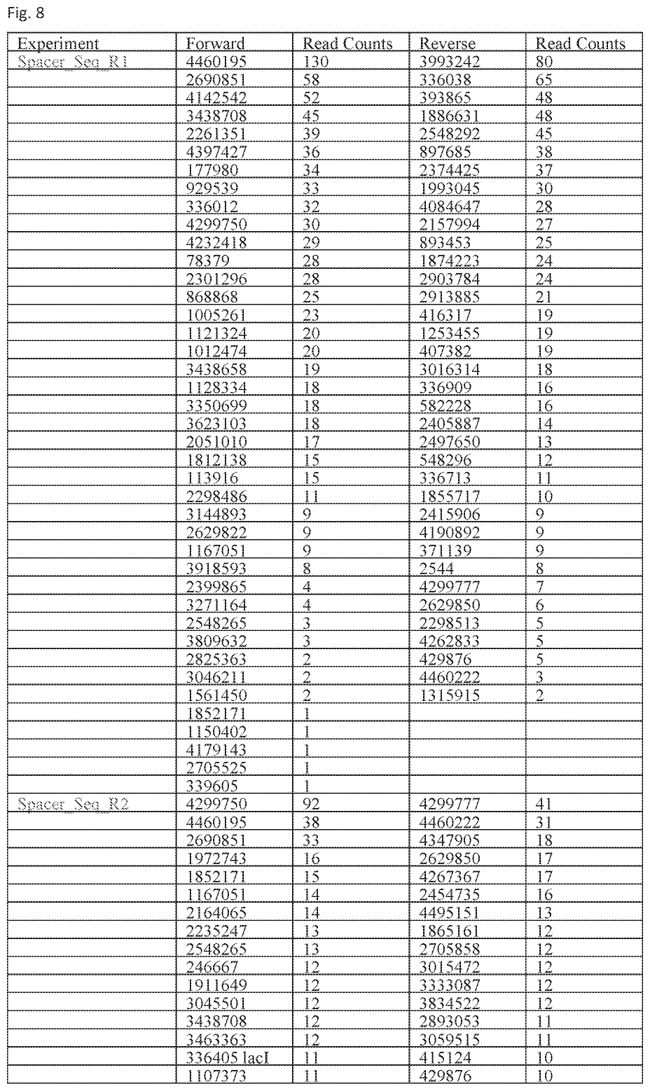

FIG. 6A depicts in schematic the Spacer-seq method or workflow which identifies hundreds of off-target spacer integration sites within the E. coli genome. As shown in FIG. 6A, (i) isolated genomic DNA containing defined spacer acquisition events is fragmented; (ii) adaptor sequences are ligated onto fragment ends. (iii) the fragments are amplified, such as by PCR, using the defined spacer sequence and adaptor sequence as primers; (iv) which results in specific enrichment of fragments containing spacer insertions, i.e, the method utilizes an additional round of PCR with a specific primer matching the defined spacer sequence to amplify only fragments of the genome that contain a new integration; (iv) the enriched fragments are then subject to high-throughput sequencing and mapping of reads to reference genome. As shown in FIG. 6B, the enrichment method was applied to the genomic fragment library previously presented in Example 1 (as well as three additional biological replicates). Only the genomic fragments that contained the psAA33 oligo protospacer sequence were specifically enriched and sequenced to identify an additional 695 unique off-target spacer integration sites (see FIG. 6B, FIGS. 7A-7B and FIG. 8).

To eliminate the potential for analyzing fragments that did not contain bonafide spacer integrations, the spacer-enrichment PCR step was performed with primers that excluded the terminal 10 basepairs of the 3′ psAA33 sequence (see FIG. 5). This allowed filtering out of fragments amplified by mispriming on regions of endogenous DNA, as they will not contain the 10 bp spacer-specific sequence that was not included in the primer. Of the Spacer-seq reads that passed this filter, about 86% of the integration sites mapped to a CRISPR locus, while the remaining reads aligned to off-target sites in the genome (about 13%) or plasmid (about 0.4%) (see FIG. 6C). Normalizing for total DNA content within the cell, off-target integrations displayed no preference between inserting into genomic or plasmid DNA (see FIG. 6D), and were typically found within the protein coding regions of non-essential genes (about 94%).

With the additional 695 off-target sites, the off-target site sequence logos were regenerated (see FIG. 6E). Comparing the new logo with the original consensus sequence generated from the 9 off-target sites identified by WGS (see FIG. 4), the same palindromic motif within the repeat is present within both logos. However, the new logo also identifies overrepresented bases near the putative leader-repeat junction (i.e. the first base of the repeat, and the first three bases of the leader), and shows no conservation for nucleotides further upstream in the leader nor in the 60 nt downstream of the repeat (see FIG. 6E).

The IR of the off-target repeat consensus (see FIG. 6E) is a perfect palindrome within bases C8 through G21 (CCNCGCGCGCGNGG (SEQ ID NO: 2)), while the endogenous E. coli CRISPR repeats have two non-palindromic bases (nucleotides C14 and A15). To test whether the perfect internal palindrome found in the off-target consensus is actually the most strongly preferred Cas1-Cas2 target site, a defined spacer acquisition assay was performed comparing the in-vivo spacer acquisition efficiency of the endogenous repeat sequence to that of a mutant repeat representing the off-target consensus sequence (i.e. containing the repeat mutations C14G and A15C). The array containing the off-target consensus repeat acquired nearly 50% more spacers than the native array (1.2±0.1% and 0.85±0.03% of all plasmid-based arrays were expanded, respectively) (see FIG. 6F). The array containing the off-target consensus repeat improves efficiency of spacer integration using E. coli Cas1-Cas2 compared to the canonical or native CRISPR repeat sequence.

### Example III

### Expression of an Off-Target Spacer Integration Product can Stimulate Primed Acquisition

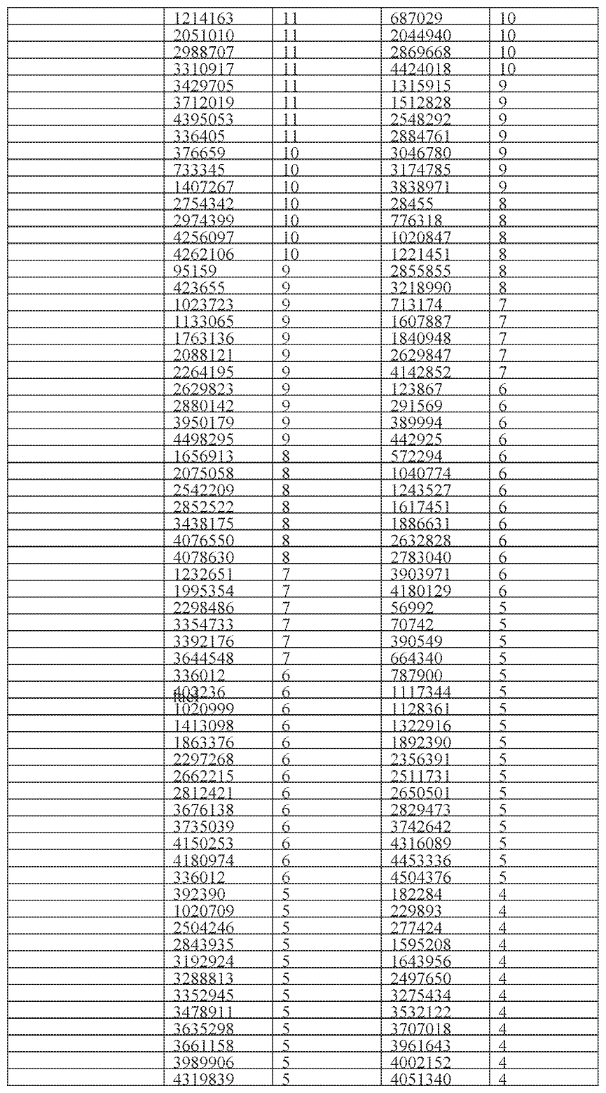

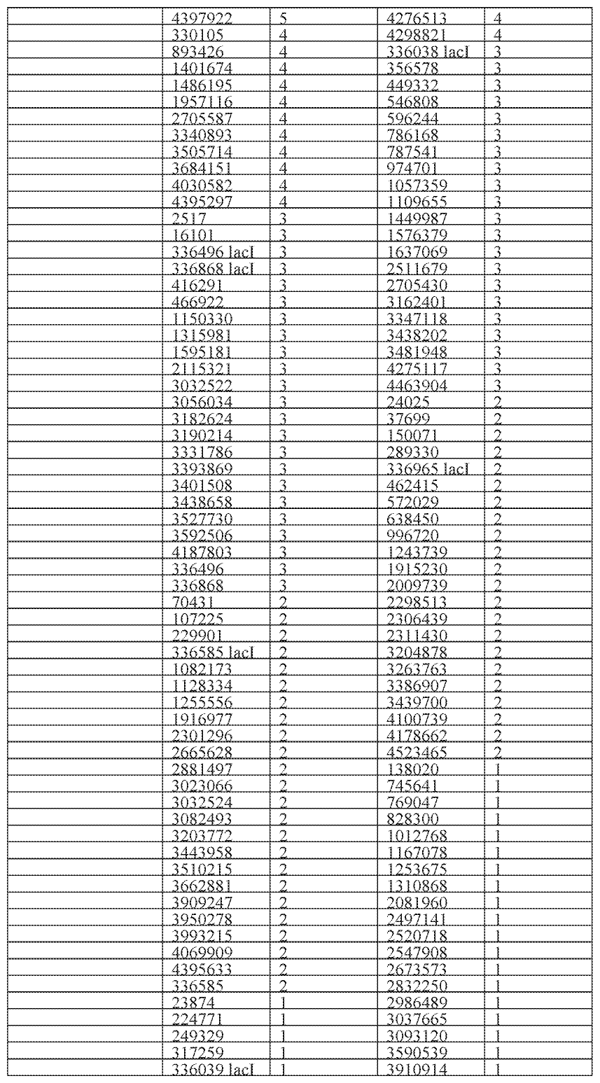

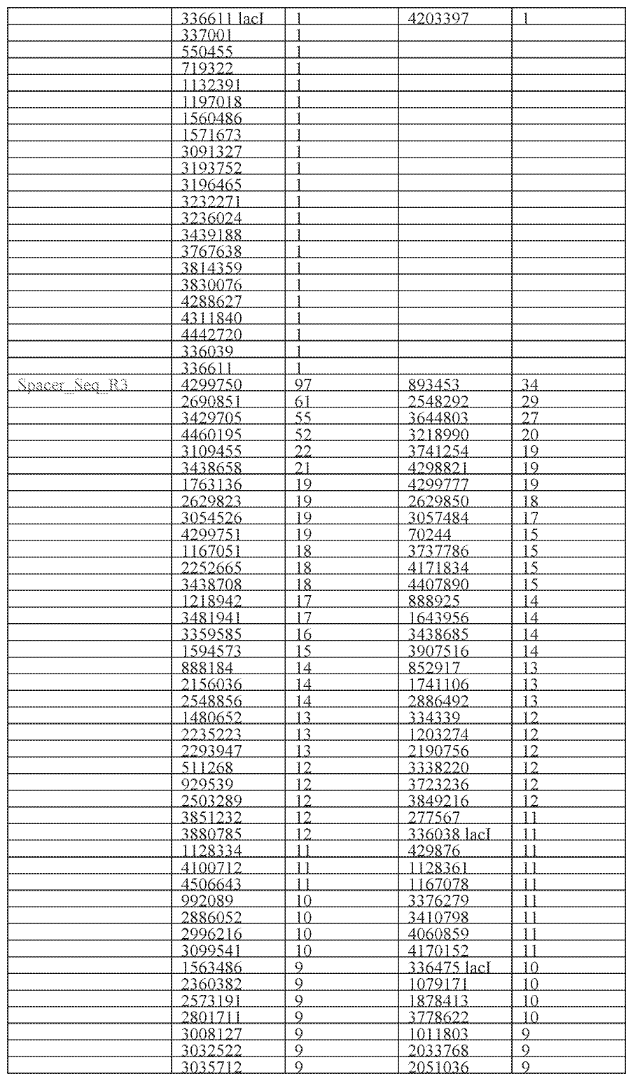

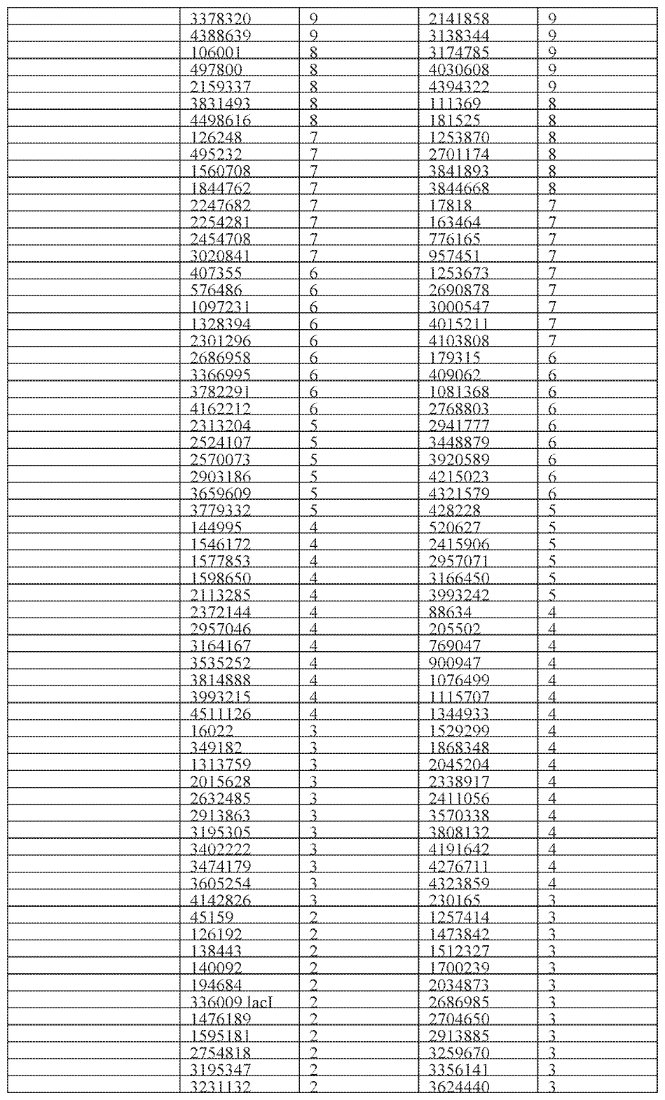

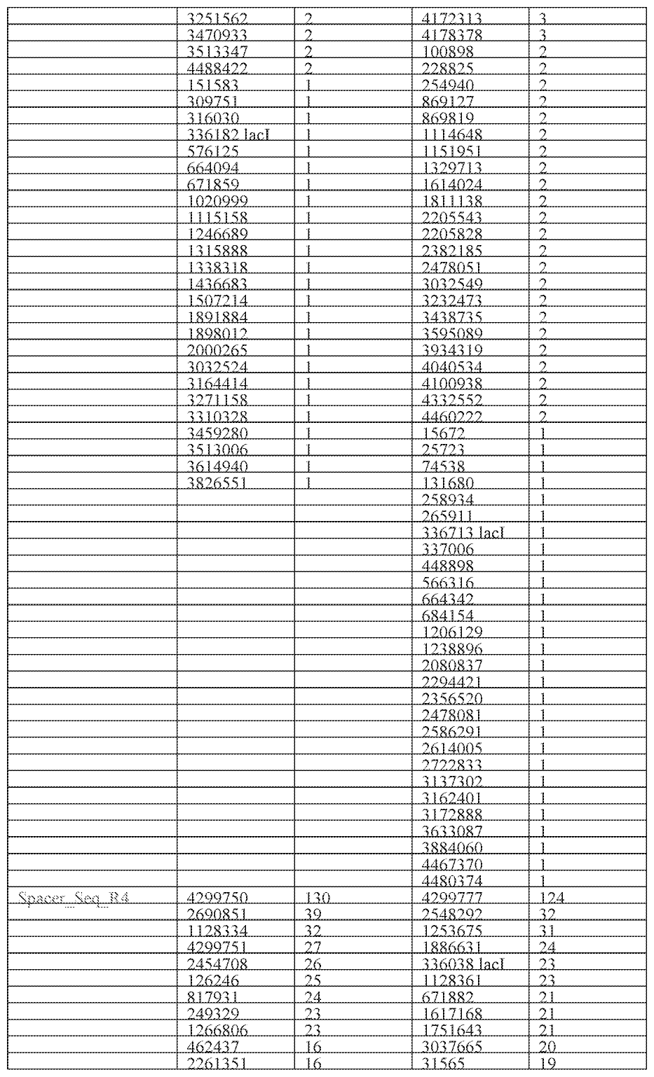

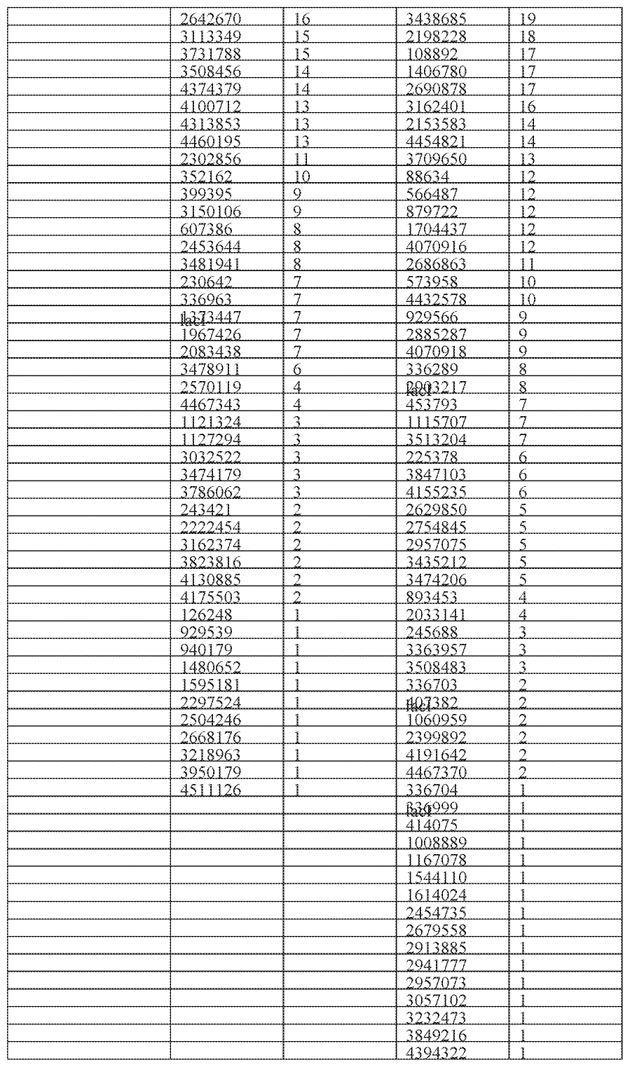

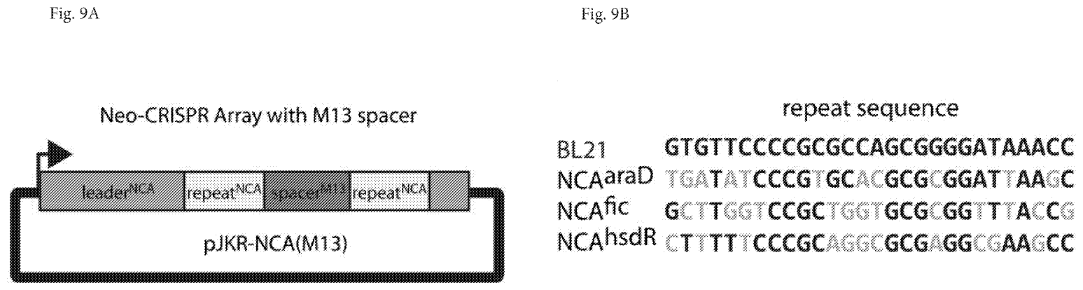

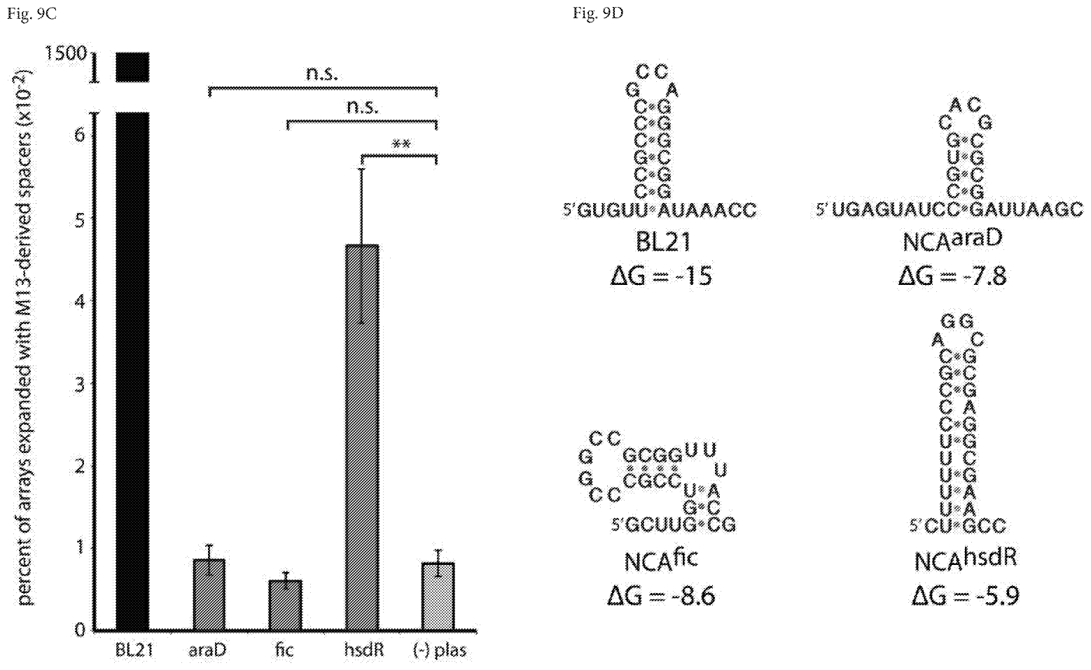

Canonical CRISPR leaders include promoter elements for the expression of crRNA transcripts, which are utilized by the Cas effector proteins for spacer-guided nuclease activity. See Marraffini, L. A. (CRISPR-Cas immunity in prokaryotes. Nature 526, 55-61. (2015) which is hereby incorporated by reference in its entirety. Most of the off-target spacer integrations characterized and described herein occur within the protein coding regions of non-essential genes (see FIG. 6E), and thus downstream of endogenous promoters. According to certain aspects, off-target integration products are transcribed upon the activity of proximal promoter elements and are expressed as functional crRNA. According to this aspect, off-target spacer acquisition activity provides or augments immunity by the genesis and expression of spacer sequences integrated into off target integration sites (“neo-CRISPR arrays”). Three off-target integration sites (sites within the araD, fic, and hsdR genes) were selected that shared substantial homology to the native CRISPR repeat, and were cloned into expression plasmids along with a spacer that matches the M13 bacteriophage genome (see FIG. 9A, FIG. 9B and FIG. 5). These plasmids were introduced into a strain of E. coli that expresses the full set of type I-E Cas genes required for adaptation and defense (BW40114). See Datsenko, K. A., Pougach. K., Tikhonov, A., Wanner. B. L., Severinov, K., and Semenova, E. Molecular memory of prior infections activates the CRISPR/Cas adaptive bacterial immunity system. Nat. Commun. 3, 945. (2012) which is hereby incorporated by reference in its entirety. These strains and bacteriophage M13 were used to perform a primed-acquisition assay. See Datsenko, K. A., Pougach, K., Tikhonov, A., Wanner, B. L., Severinov, K., and Semenova, E. Molecular memory of prior infections activates the CRISPR/Cas adaptive bacterial immunity system. Nat. Commun. 3, 945. (2012) and Conrath, U., Beckers, G. J., Langenbach, C. J., and Jaskiewicz. M. R. Priming for enhanced defense. Annu. Rev. Phytopathol. 53, 97-119. (2015) each of which is hereby incorporated by reference in its entirety. Efficient acquisition of new spacers during a phage challenge of this system requires the presence of a pre-existing spacer that matches the phage genome and enables “priming” of the acquisition process. Thus, the plasmid-based neo-CRISPR arrays containing the M13 spacer should enhance the acquisition of phage-derived spacers during the M13 phage challenge if the neo-arrays express functional crRNA. The results of the primed-acquisition assay are shown in FIG. 9. Although two of the neo-CRISPR arrays (NCA^{araD} and NCA^{fic}) did not stimulate additional spacer acquisitions relative to a negative control lacking a plasmid-based array (0.009±0.002%, 0.006±0.001%, and 0.008±0.002% M13-expanded arrays, respectively), cells expressing NCA^{hsdR} acquired about 5 fold more M13-derived spacers within their genomic arrays over the background level (0.05±0.009%). According to certain aspects a method is provided wherein an off-target integration event leads to the expression of functional crRNA, thereby providing a selective advantage during phage infection.

The secondary structure of crRNA is important to its biogenesis and activity in DNA interference. See Charpentier, E., Richter. H., van de Oost, J., and White, M. F. Biogenesis pathways of RNA guides in archaeal and bacterial CRISPR-Cas adaptive immunity. FEMS Microbiol Rev. 39(3):428-41 (2015) which is hereby incorporated by reference in its entirety. Specifically, the type I-E crRNA repeat forms a 7 bp hairpin motif that serves a “molecular handle” and is crucial for efficient processing of the pre-crRNA into mature crRNA (see FIG. 9D, BL21 repeat). See Beloglazoval, N. et al. CRISPR RNA binding and DNA target recognition by purified Cascade complexes from Escherichia coli. Nucleic Acids Res. 43(1):530-43. (2015) which is hereby incorporated by reference in its entirety. The presence of any structural motifs or secondary structures within the three different neo-CRISPR array repeats that might explain differences in NCA crRNA efficiency were predicted using mFold. See Zuker, M. Mfold web server for nucleic acid folding and hybridization prediction. Nucleic Acids Res. 31 (13), 3406-15, (2003) which is hereby incorporated by reference in its entirety. While all three of the neo-CRISPR arrays have the propensity to form hairpin motifs, NCA^{hsdR} forms the longest hairpin structure (up to 11 bp) (see FIG. 9D) which may result in the higher level of NCA^{hsdR} activity in the primed acquisition assay.

### Example IV

### Spontaneous CRISPR Loci Generation by Non-Canonical Spacer Integration

The present disclosure contemplates that non-canonical off-target integrations can occur within bacterial chromosomes at locations resembling the native CRISPR locus by characterizing hundreds of off-target integration locations within Escherichia coli (E. coli). Embodiments are directed to combing existing CRISPR databases and available genomes for evidence of off-target integration activity, considering whether such promiscuous Cas1-Cas2 activity could play an evolutionary role through the genesis of neo-CRISPR loci. This search uncovered several putative instances of naturally occurring off-target spacer integration events within the genomes of Yersinia pestis (Y. pestis) and Sulfolobus islandicus (S. islandicus). The present disclosure is instrumental in understanding alternative routes to CRISPR array genesis and evolution, as well as in the use of spacer acquisition in technological applications.

In-vitro, spacer integration events occur outside of canonical CRISPR arrays (“off-target” sites) with relatively high frequency, albeit with a lower occurrence in the presence of IHF (Nuñez, J. K., Bai, L., Harrington, L. B., Hinder, T. L. & Doudna, J. A. CRISPR immunological memory requires a host factor for specificity. Mol. Cell 62, 824-833 (2016), K. Nuñez, A. S. Lee, A. Engelman, J. A. Doudna, Integrase-mediated spacer acquisition during CRISPR-Cas adaptive immunity. Nature 519, 193-198 (2015)). Even so, this is surprising considering that specific integration into the 5′ end of the array is essential for robust immunity (McGinn, J., and Marraffini, L. A. CRISPR-Cas systems optimize their immune response by specifying the site of spacer integration. Mol Cell 16, 30517-2 (2016)) and overall genomic integrity (Wright, A V., Doudna, J A. Protecting genome integrity during CRISPR immune adaptation. Nat. Struct and Mol. Biol. (10):876-883 (2016))

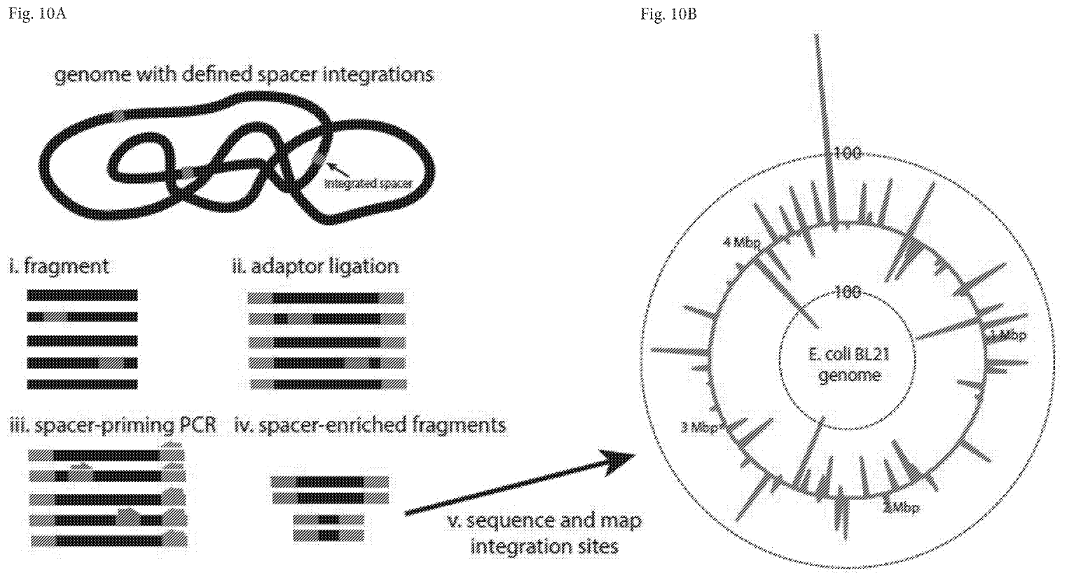

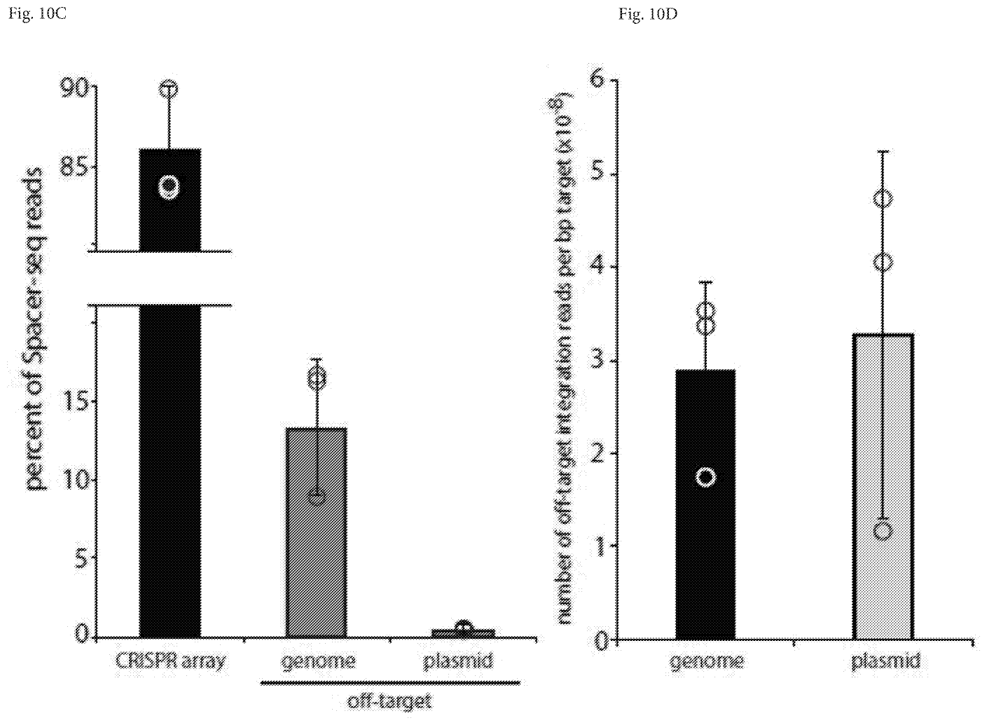

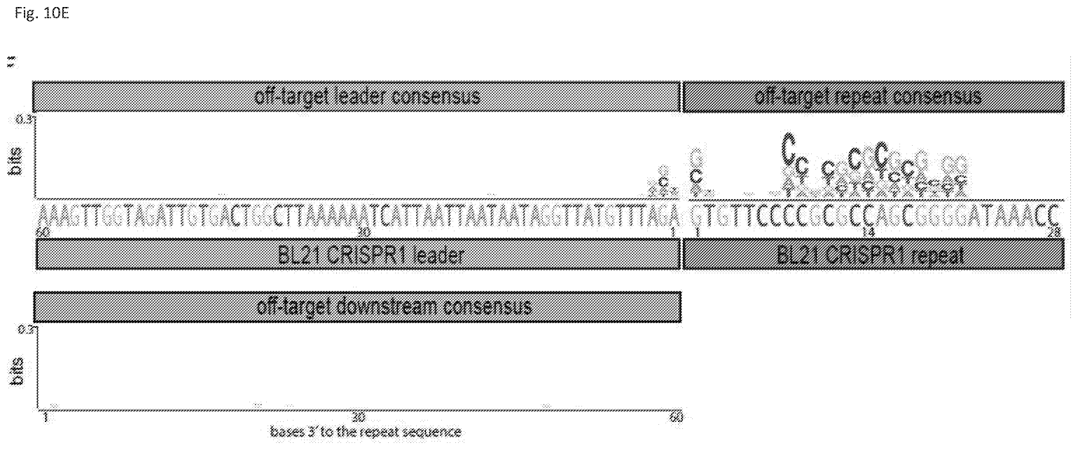

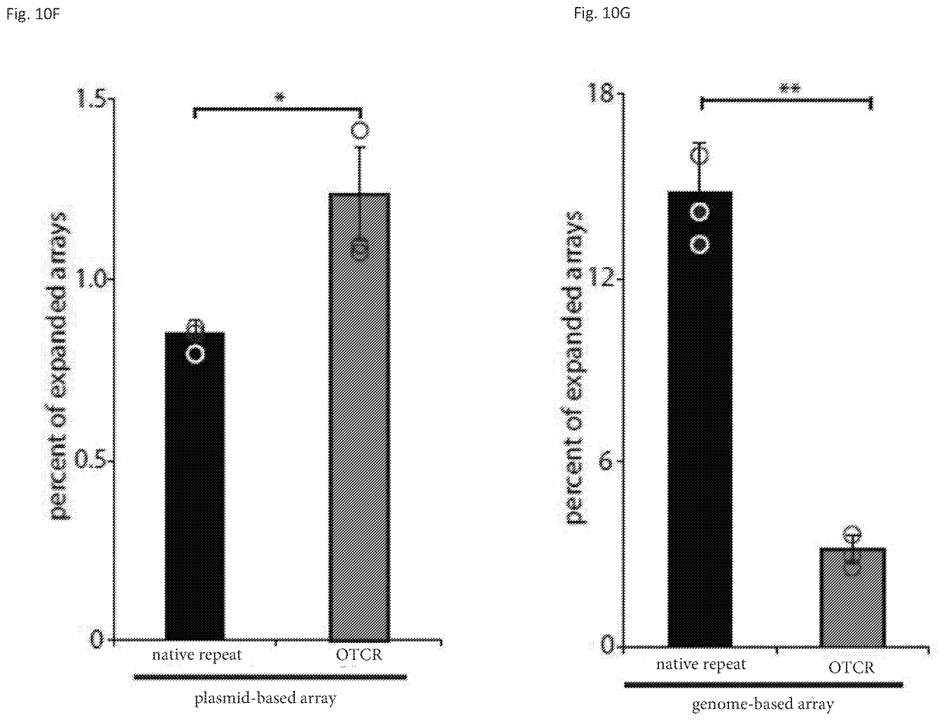

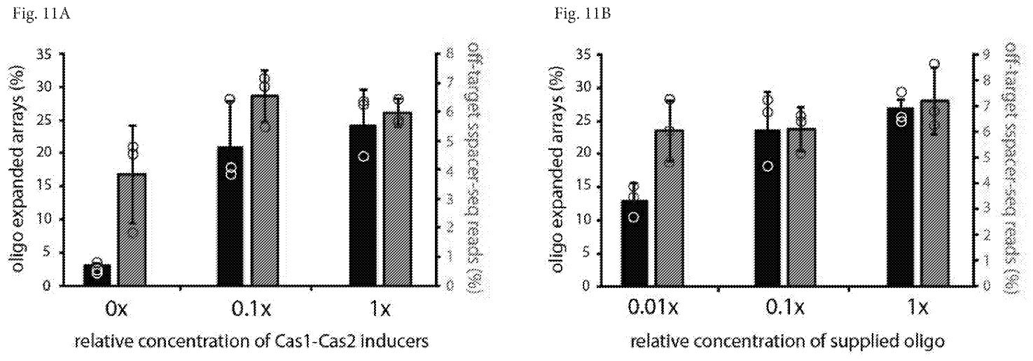

Methods of the present disclosure are directed to radically expand the number of off-target sites that can be identified without having to continually sequence the genome to extreme depths. In one embodiment, a method is developed to target our sequencing to spacer integration sites, termed Spacer-seq (FIG. 10A). After prepping whole-genome libraries, the Spacer-seq approach utilizes an additional round of PCR with a specific primer matching the defined spacer sequence to amplify only fragments of the genome that contain a new integration. Applying Spacer-seq to the genomic fragment library previously presented in FIGS. 2A-2C (as well as three additional biological replicates), the method specifically enriched and sequenced only the genomic fragments that contained the psAA33 oligo protospacer sequence, and discovered an additional 695 unique off-target spacer integration sites (FIG. 10B, FIGS. 7A-7B, and FIG. 8). To eliminate the potential for analyzing fragments that did not contain bona fide spacer integrations, the spacer-enrichment PCR step was performed with primers that excluded the terminal 10 basepairs of the 3′ psAA33 sequence (FIG. 3). This method allowed filtering out fragments amplified by mispriming on regions of endogenous DNA, as they would not contain the 10 bp spacer-specific sequence that was excluded in the primer. Of the Spacer-seq reads that passed this filter. ˜86% of the integration sites mapped to a CRISPR locus, while the remaining reads aligned to off-target sites in the genome (˜13%) or plasmid (˜0.4%) (FIG. 10C). Normalizing for total DNA content within the cell, off-target integrations displayed no preference between inserting into genomic or plasmid DNA (FIG. 10D), and were typically found within the protein coding regions of non-essential genes (˜94%). Additionally, to investigate how threshold effects may influence the frequency of off-targeting, the experiment was replicated with decreasing levels of Cas1-2 induction and oligo protospacer. It was observed that, while decreasing the concentration of oligo by up to 10-2 or not inducing Cas1-2 expression substantially lowers overall acquisition efficiency, no significant effect is similarly observed in the overall ratio of on:off target integrations (FIGS. 11A-11B).

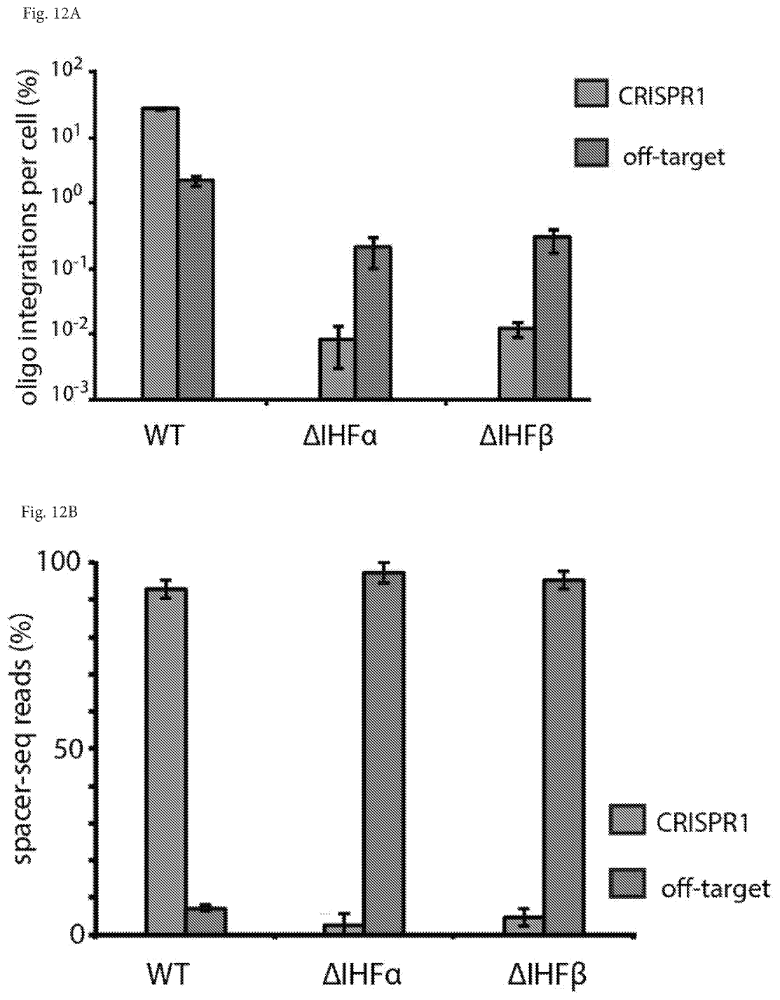

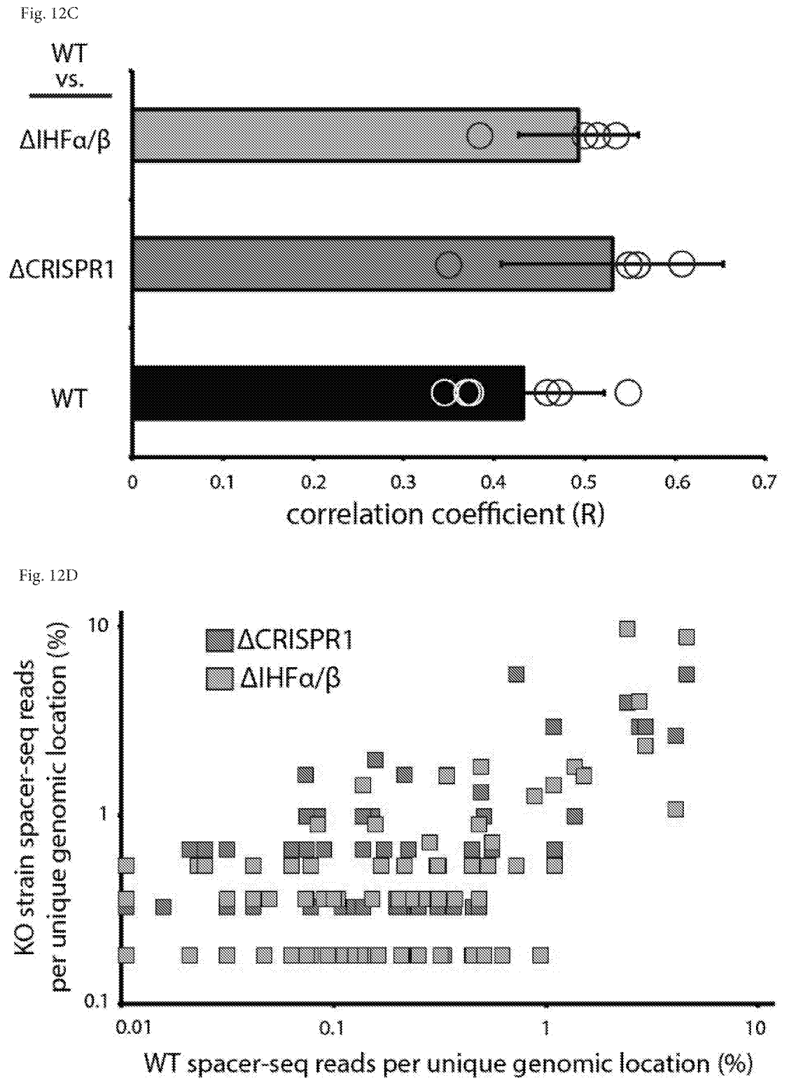

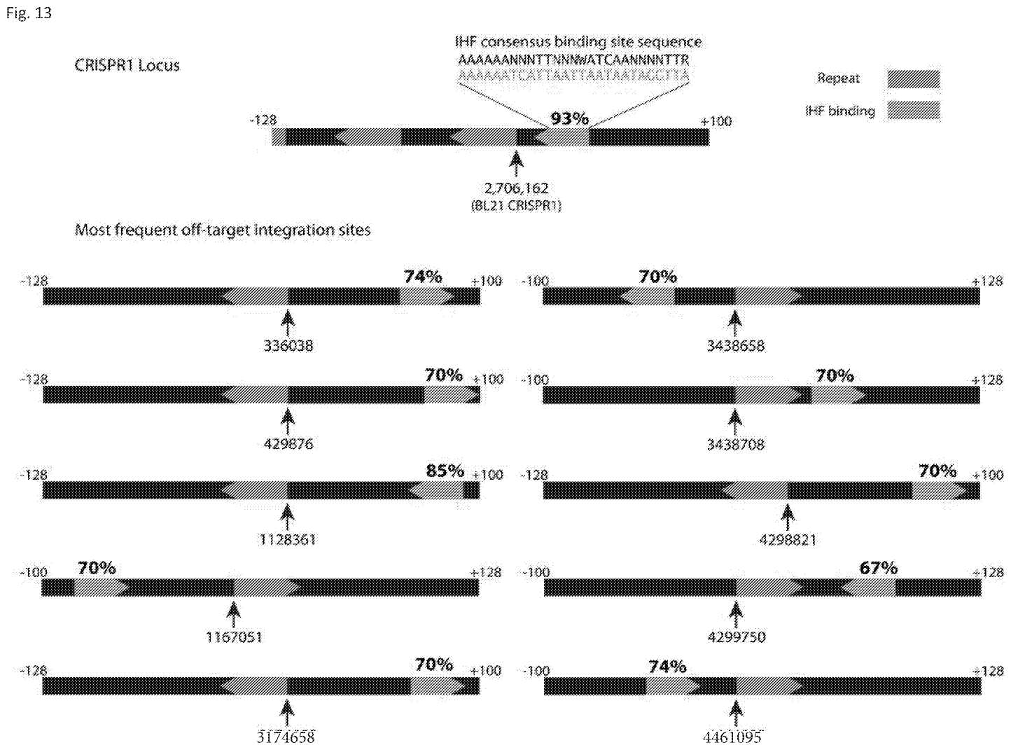

With the additional 695 off-target sites, the off-target site sequence logos were regenerated (FIG. 10E). Comparing the new logo with the original consensus sequence generated from the 9 off-target sites identified by WGS (FIG. 4), the same palindromic motif within the repeat is present. However, the new logo also identifies overrepresented bases near the putative leader-repeat junction (i.e., the first base of the repeat, and the first three bases of the leader) (Rollie, C., Schneider, S., Brinkmann. A. S., Bolt, E L., and White, M F. Intrinsic sequence specificity of the Cas1 integrase directs new spacer acquisition. eLife 4:e08716. (2015)), and shows no conservation for nucleotides further upstream in the leader nor in the 60 nt downstream of the repeat (FIG. 10E). This result is surprising considering that an IHF-binding motif located in the native leader sequence upstream of the first repeat was previously found to be essential for integrations into the canonical CRISPR array in-vivo (Nuñez. J. K., Bai. L., Harrington. L. B., Hinder, T. L. & Doudna. J. A. CRISPR immunological memory requires a host factor for specificity. Mol. Cell 62, 824-833 (2016)). DSA experiments were therefore performed and Spacer-seq on knockout strains lacking the alpha or beta subunit of IHF (an obligate heterodimer) to determine what effect IHF has in off-target insertions. The IHF knockout strains had substantially reduced integration efficiencies (˜10³ fold reduction) into the native CRISPR1 array (on-target), while overall off-target integration rates only decreased ˜10-20 fold, with ˜95% of all spacer integrations going into off-target regions of the genome (FIGS. 12A-12B). The locations of the off-target sites found in the IHF knockouts strains were then compared to those of the WT and similar distribution profiles were observed (correlation coefficient of R=0.499±0.07 for IHF knockouts vs. WT, compared to R=0.43±0.09 for WT vs WT experimental replicates), with the most frequent off-target sites being consistent across all strains (FIGS. 12C-12D). These results suggest that the presence of IHF increases the efficiency of both on and off-target integration activity, although off-target activity is less dependent on the presence of IHF overall. To better understand these results, potential IHF binding motifs near the ten most prevalent off-target locations across all datasets and strains were searched for. It was found that all these sites had regions within 100 nt of the off-target repeat that shared at least 67% identity to the IHF consensus binding motif (FIG. 13), supporting our results that IHF enhances the rates of both on and off-target integration events. Previous in-vitro experiments have demonstrated that, even in the absence of IHF, efficient spacer integration into supercoiled plasmid-based CRISPR arrays can still occur (Nuñez, J. K., Bai. L., Harrington, L. B., Hinder, T. L. & Doudna. J. A. CRISPR immunological memory requires a host factor for specificity. Mol. Cell 62, 824-833 (2016)). It was thus determined what effect the addition of a plasmid including a canonical CRISPR array would have in the genomic contexts of the IHF knockout strains during DSA. Spacer-seq results from these experiments demonstrated that the addition of a multicopy plasmid-based array reduces the frequency of off-target events into the genome from ˜95% down to ˜35%, with ˜60% of integrations now going into the plasmid-based array on-target, even in the absence of IHF (FIG. 14). Meanwhile, on-target integration frequency into the genomic array remained unchanged at ˜5% of all Spacer-seq reads.

The present disclosure further contemplates that if removing the only active CRISPR array in the BL21 genome (CRISPR1) might influence the distribution of off-target sites elsewhere in the genome. In one embodiment, a CRISPR1 deletion strain was created by deleting the entire CRISPR1 array and leader region from the BL21-AI genome, and performed DSA followed by Spacer-seq. The locations of the off-target sites found in the CRISPR1 deletion strain were then compared to those of the WT and similar distribution profiles was observed (correlation coefficient of R=−0.53±0.12 for the CRISPR1 deletion vs. WT, compared to R=0.43±0.09 for WT vs WT experimental replicates), with the most frequent off-target sites again being consistent across all strains (FIGS. 12C-12D).

Curiously, the IR of the off-target repeat consensus (FIG. 10E) is a perfect palindrome within bases C8 through G21 (CCNCGCGCGCGNGG (SEQ ID NO: 2)), while the endogenous E. coli CRISPR repeats have two non-palindromic bases (nucleotides C14 and A15). The off-target perfect palindrome logo is similar to a logo generated from aligning all the repeats associated with the Type I-E repeat class, as previously shown (Kunin, V., Sorek, R. and Hugenholtz, P. Evolutionary conservation of sequence and secondary structures in CRISPR repeats. Genome Biol. 8:R61 (2007)). To test whether the perfect internal palindrome found in the off-target consensus is actually the most strongly preferred Cas1-Cas2 target site, a defined spacer acquisition assay was performed by comparing the in-vivo spacer acquisition efficiency of the endogenous repeat sequence to that of a mutant repeat representing the off-target consensus sequence (i.e., containing the repeat mutations C14G and A15C), termed the “off-target consensus repeat” or OTCR. It was found that the array containing the OTCR acquired nearly 50% more spacers than the native array (1.2±0.1% and 0.85±0.03% of all plasmid-based arrays were expanded, respectively) (FIG. 10F).

In one embodiment, a modified BL21-AI strain was created in which its native CRISPR1 locus was replaced with a minimal version of the array containing the OTCR, to see whether these results were specific to a plasmid-based array. Engineering strains with enhanced spacer acquisition activity would also be useful in molecular recording applications (Shipman, S. L., Nivala. J., Macklis, J. D., Church, G. M. Molecular recordings by directed CRISPR spacer acquisition. Science 353(6298) (2016), Shipman, S L., Nivala. J., Macklis, J D., and Church, G M. CRISPR-Cas encoding of a digital movie into the genomes of a population of living bacter. Nature 547, 345-349 (2017)). In one embodiment, a synthetic CRISPR array containing the first 100 nt of the native CRISPR1 leader upstream of the OTCR sequence was designed and integrated into the BL21-AI CRISPR1 deletion strain that was previously constructed. However, in DSA experiments quantifying oligo acquisition efficiencies, the OTCR strain actually displayed lower acquisition rates compared to those of the WT BL21-AI (FIG. 10G). This finding conflicts with the plasmid-based array results (FIG. 10F), suggesting that array activity is context dependent and that additional regions outside of the first repeat and leader might affect acquisition efficiency. For instance, in modifying the first the repeat, subsequent repeats were also deleted. It is contemplated that the presence of many repeats within an array may help recruit Cas1-Cas2 localization to the CRISPR locus.

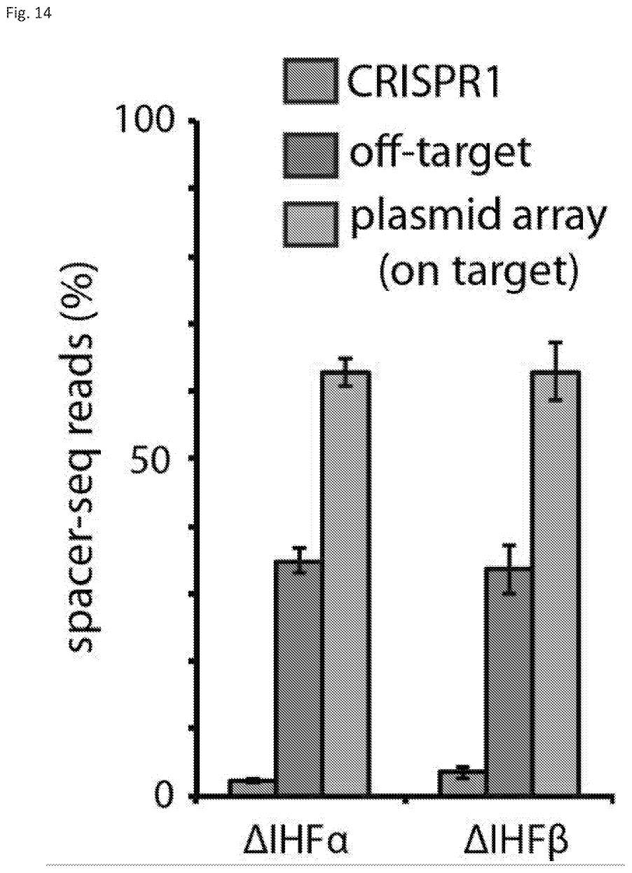

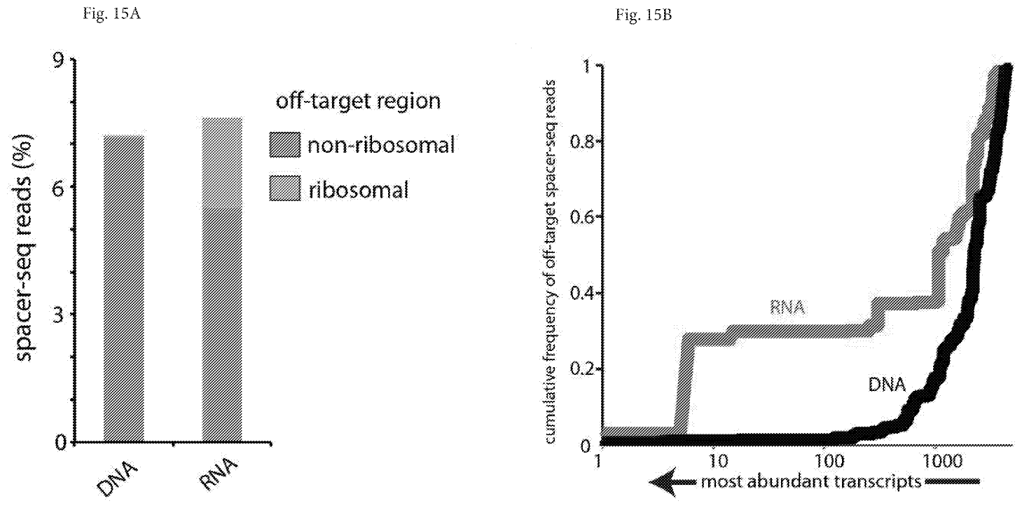

Canonical CRISPR leaders include promoter elements for the expression of crRNA transcripts, which are utilized by the Cas effector proteins for spacer-guided nuclease activity (Marraffini, L. A. (CRISPR-Cas immunity in prokaryotes. Nature 526, 55-61. (2015)). Most of the off-target spacer integrations that were characterized occur within the protein coding regions of non-essential genes, and thus downstream of endogenous promoters. This is not surprising given the high density of genes in bacterial genomes. This observation suggests the possibility that these off-target integration products could be transcribed, dependent upon the activity of proximal promoter elements. Therefore, the present disclosure contemplates whether the expression of off-target integration products within cellular transcripts can be detected. In one embodiment, Spacer-seq on cDNA derived from the total RNA isolated from cultures of BL21-AI cells following DSA was performed. Sequencing results from these experiments confirmed the expression of off-target integration products, with the overall frequency of off-target reads within transcripts similar to the levels found in the genome (FIG. 15A). These RNA Spacer-seq reads mapped to the most abundant cellular transcripts (FIG. 15B), as further evidenced by enrichment for off-target sites within ribosomal operons (FIG. 15A).

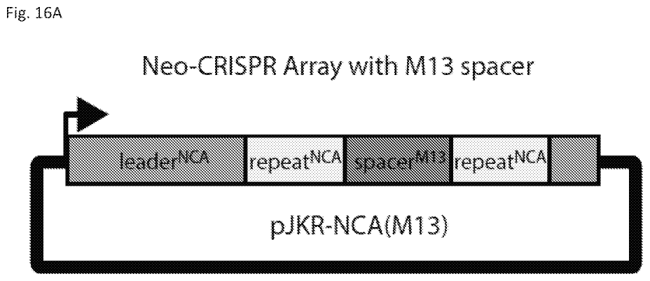

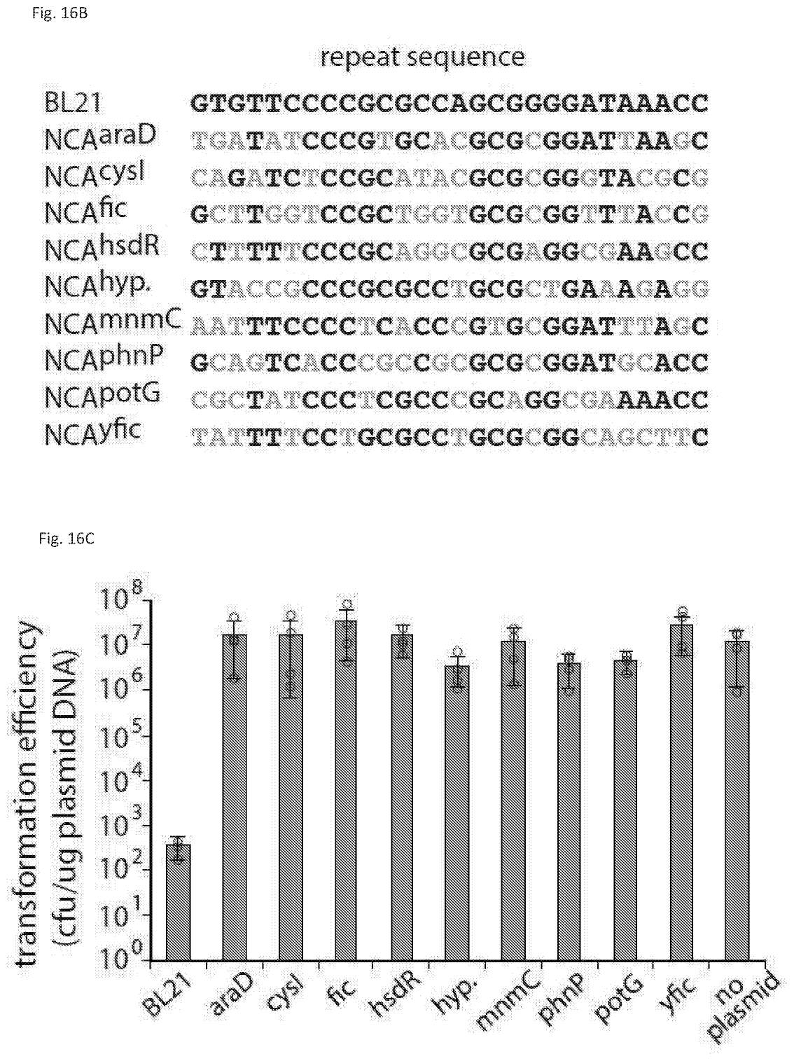

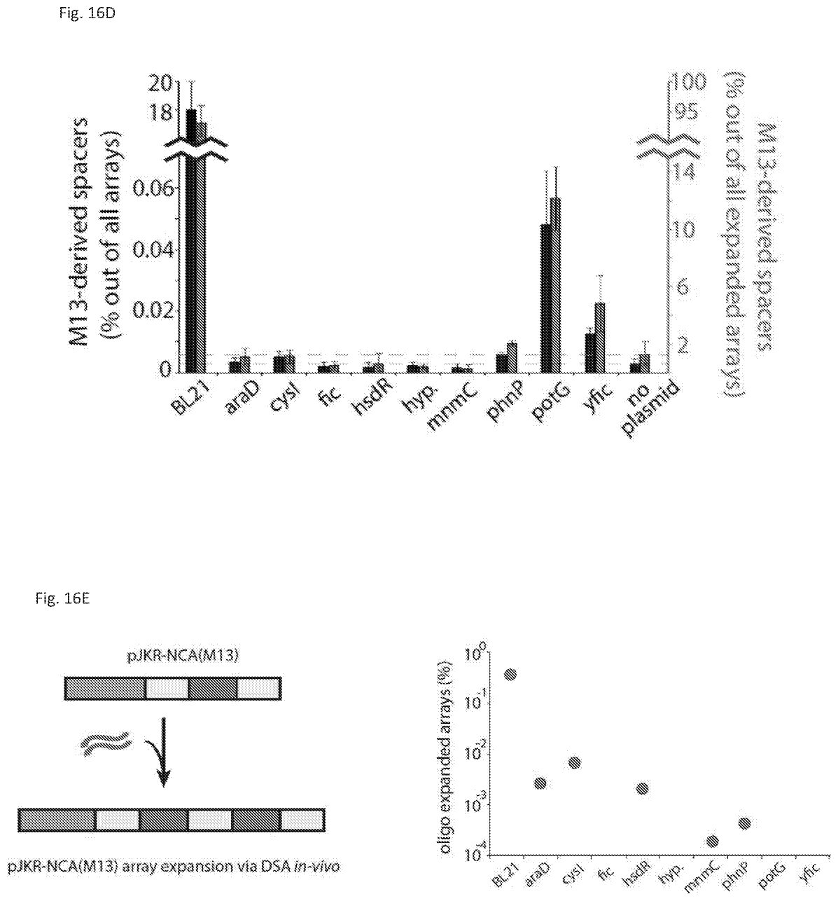

After confirming that off-target integration products retain the potential to be expressed, it was contemplated whether some of these transcripts could function as crRNA in defense. If so, it would imply that off-target spacer acquisition activity has the potential to augment immunity by the incidental genesis and expression of “neo”-CRISPR arrays (NCAs). In some embodiments, 10 off-target integration sites that were discovered by Spacer-seq (sites within the araD, cysI, fic, hsdR, mnmC, phnP, potG, and yfic genes, in addition to a site within an unnamed hypothetical protein “hyp.”) that share considerable homology to the native CRISPR repeat were selected, and cloned them into expression plasmids along with a spacer that matches the M13 bacteriophage genome (FIGS. 16A-16B and FIG. 3). These plasmids were introduced into a strain of E. coli that expresses the full set of type I-E Cas genes required for adaptation and defense (BW40114) (Datsenko, K. A., Pougach, K., Tikhonov, A., Wanner. B. L., Severinov, K., and Semenova, E. Molecular memory of prior infections activates the CRISPR/Cas adaptive bacterial immunity system. Nat. Commun. 3, 945. (2012)). First, these strains were used to see whether any of the cloned plasmid-based NCAs could function in direct interference through a plasmid interference assay (Kuznedelov, K., et al. Altered stoichiometry Escherichia coli Cascade complexes with shortened CRISPR RNA spacers are capable of interference and primed adaptation. Nucleic Acids Res. 44(22), 10849-10861 (2016)) by attempting to transform an additional plasmid that also contained the M13 spacer target sequence into cultures expressing the NCA and Cas proteins. If the NCAs are functional, they should reduce the efficiency of this transformation relative to a negative control plasmid that doesn't contain a matching protospacer target. The results of this interference assay are shown in FIG. 16C. While a strain expressing the canonical BL21 array and M13 spacer reduced the transformation efficiency by over four orders of magnitude compared to the negative control, none of the NCA strains demonstrated a significant effect on transformation efficiency (FIG. 16C).

While plasmid interference is the most direct test of a functional CRIPSR system, it is not the most sensitive. Recently, it was shown that a more sensitive in-vivo test of crRNA function is a primed-acquisition assay (Kuznedelov, K., et al. Altered stoichiometry Escherichia coli Cascade complexes with shortened CRISPR RNA spacers are capable of interference and primed adaptation. Nucleic Acids Res. 44(22), 10849-10861 (2016)). Briefly, “priming” is the efficient acquisition of new spacers during a phage challenge of this system stimulated by a pre-existing spacer matching the phage genome that enhances the acquisition of additional phage spacers. Thus, the plasmid-based NCAs containing the M13 spacer should enhance the acquisition of phage-derived spacers during an M13 phage challenge if the neo-arrays express functional crRNA. The results of the primed-acquisition assay are shown in FIG. 16D. Although the majority of the neo-CRISPR arrays did not stimulate additional spacer acquisitions relative to a negative control lacking a plasmid-based array, cells expressing NCA^{potG} acquired ˜16-fold more M13-derived spacers compared to background (0.048±0.02% versus 0.003±0.002%, respectively). Additionally, the NCA^{potG} strain had an increased bias for M13-derived spacers within its newly acquired spacer population compared to background (12.1±2.2% versus 1.3±0.9%). Although these frequencies are well below the rates observed for the native BL21 array strain, only a small fraction of the hundreds of possible NCA sequences have been tested, and thus one can envision additional off-target sequences with greater crRNA functionality. Even still, these results support a model in which an off-target integration event could lead to the expression of at least semi-functional crRNA.

A key feature of CRISPR-Cas immunity is the ability to store multiple spacers within a single locus. This is achieved through iterative integration events overtime into the same leader-repeat site, which is inherently preserved following integration and repeat duplication. To investigate whether NCA sites can also undergo multiple expansions beyond the original off-target event, DSA was performed on the strains containing the plasmid-based NCAs. Deep sequencing of the NCA loci following DSA revealed that five out of the nine NCAs could be expanded with an additional spacer, albeit at orders of magnitude less efficiency than the canonical array (FIG. 16E).

Having demonstrated in the presently disclosed model system that off-target spacer integration by Cas1-2 occurs in-vivo at CRISPR repeat-like sequences within the E. coli genome, it was asked whether evidence for natural off-target activity in other species could be found by using existing genomic databases. In one embodiment, the literature for bacterial and archaeal species that have well-annotated phylogeny, published indications of active CRISPR-Cas systems, and available whole genome sequences was searched. As described previously herein, the CRISPRdb (Grissa, I., Vergnaud, G., and Pourcel, C. The CRISPRdb database and txools to display CRISPRs and to generate dictionaries of spacers and repeats. BMC Bioinformatics. 8:172. (2007)) online database for related species and strains that had dissimilar numbers of CRISPR loci was combed. The search yielded support for off-target activity or “neo-CRISPR genesis” (FIG. 1) within related strains of two different microbial species with active CRISPR-Cas systems. Y. pestis and S. islandicus. The first we will describe are those of Y. pestis.

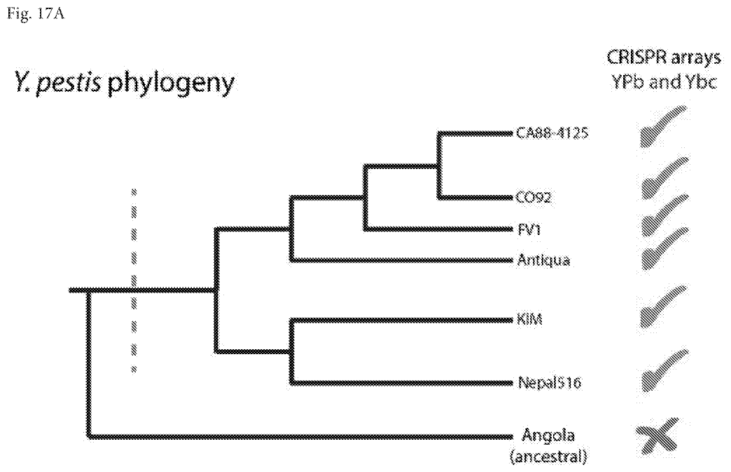

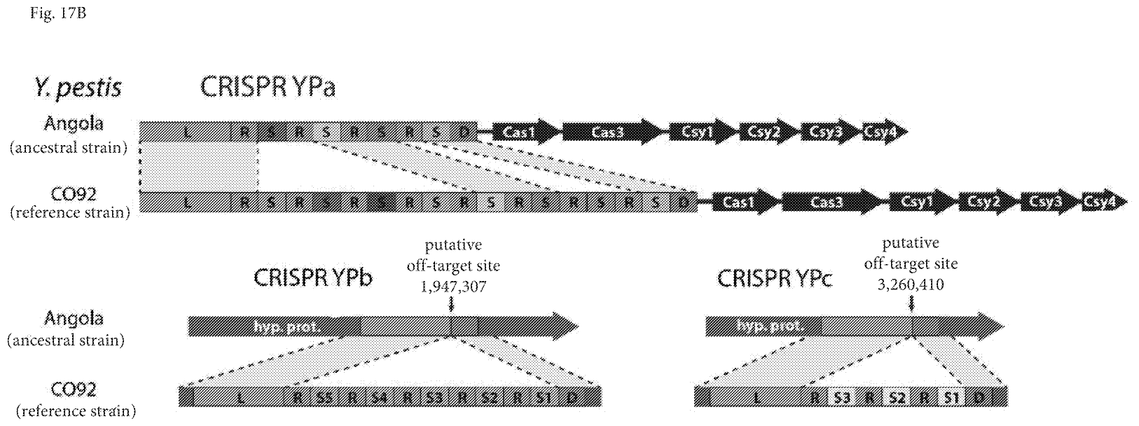

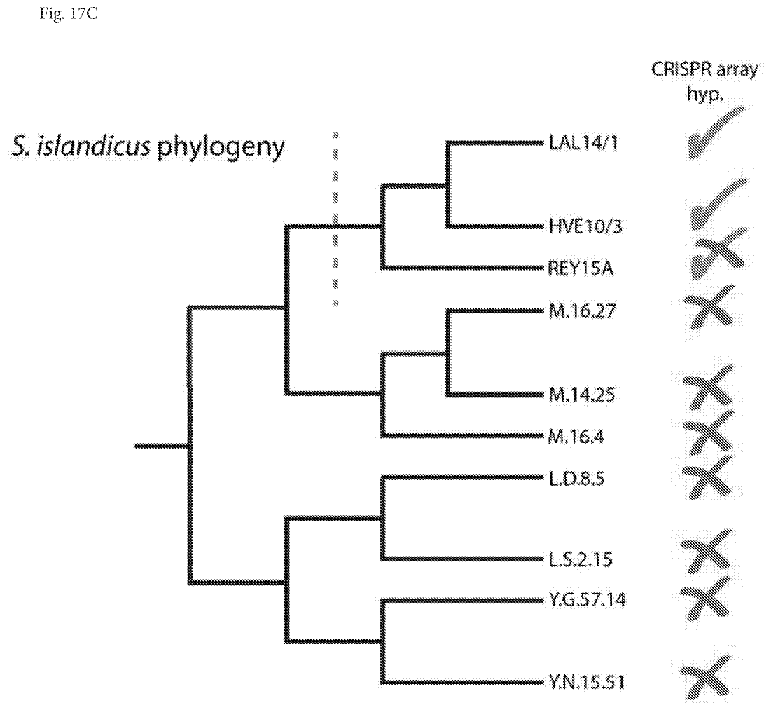

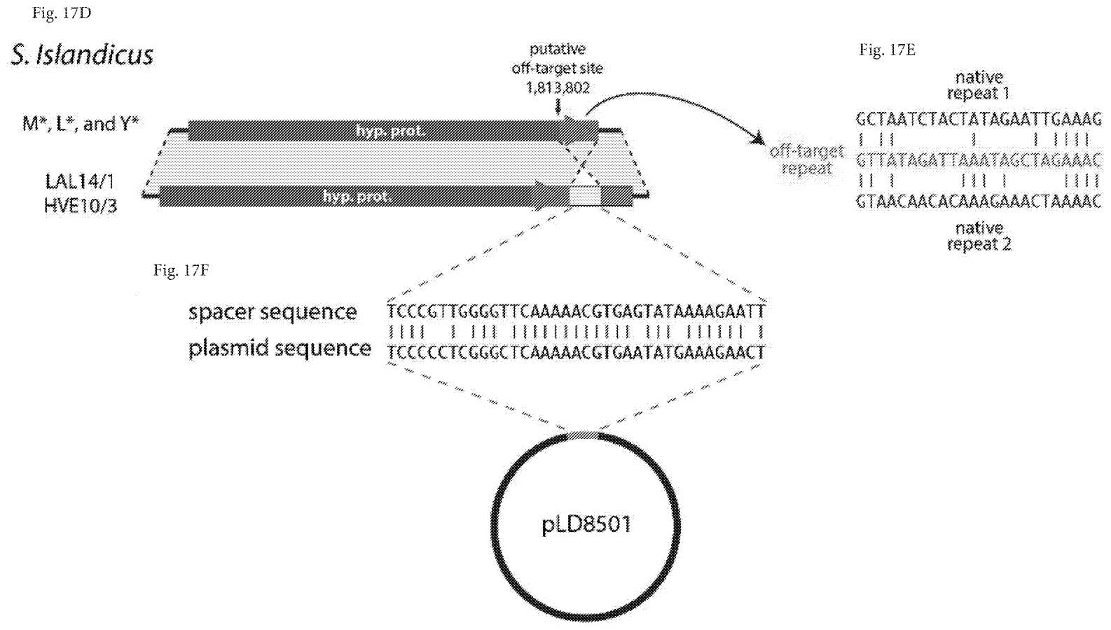

Due to its potential as a human pathogen, Y. pestis phylogeny has been heavily studied, with many strains of the species whole-genome sequenced (Barros M P S., et al. Dynamics of CRISPR Loci in Microevolutionary Process of Yersinia pestis Strains. PLoS ONE 9(9): e108353. (2014)). One of the modern Y. pestis strains, CO92, is typically used as the reference strain (Eppinger, M., et al. Genome sequence of the deep-rooted Yersina pestis strain angola reveals new insights into the evolution and pangenome of the plague bacterium. J. Bacteriol. 192:6, 1685-1699. (2010)). All but one strain of Y. pestis have three active CRISPR loci (YPa, YPb, and YPc), and only one of these loci is proximal to a set of Cas genes (YPa) (Barros M P S., et al. Dynamics of CRISPR Loci in Microevolutionary Process of Yersinia pestis Strains. PLoS ONE 9(9): e108353. (2014)) (FIGS. 17A-17B). The exception to this is the Angola strain, which only has the YPa CRISPR-Cas locus, and is considered an ancient strain in the Y. pestis lineage (Eppinger, M., et al. Genome sequence of the deep-rooted Yersina pestis strain angola reveals new insights into the evolution and pangenome of the plague bacterium. J. Bacteriol. 192:6, 1685-1699. (2010)). In place of the other two loci are single degenerate repeats and accompanying leader regions, with both loci lying within hypothetical protein coding regions. It is postulated that arrays YPb and YPc are the result of off-target integration events that became fixed in strains following the divergence from the ancient Angola strain through the process of neo-CRISPR genesis.

The second example of native off-target spacer integration that was found was in three closely related strains of the hyperthermophilic archaeal species S. Islandicus: LAL14/1, HVE10/3, and REY15A (Jaubert, C. Genomics and genetics of Sulfolobus islandicus LAL14/1, a model hyperthermophilic archaeon. Open Biol. 3(4), 130010 (2013)) (FIG. 17C). While all ten strains within this species possess multiple active CRISPR-Cas systems (Gudbergsdottir, S., et al. Dynamic properties of the Sulfolobus CRISPR/Cas and CRISPR/Cmr systems when challenged with vector-borne viral and plasmid genes and protospacers. Mol Microbiol. 79(1) 35-49. (2011)), only these three strains contain a region with a 37 nt spacer flanked by 24 nt repeats following the end of a hypothetical ABC transporter related protein (FIG. 17D). The other seven genomes only contain a single copy of the repeat. Intriguingly, the repeat is the same size as and shares sequence homology with the other two confirmed CRISPR array repeat sequence types found within the species (FIG. 17E). The spacer length is also typical for these CRISPR types. Further, a blastn search of the spacer sequence uncovered a partial match with a known S. islandicus plasmid (pLD8501) that is not present in these strains (Reno, M L., Held, N L., Fields. C J., Burke, P V., and Whitaker, R J. Biogeography of the Sulfolobus islandicus pan-genome. PNAS. 106:21. (2009)) (FIG. 17F). This is significant because canonical CRISPR spacers often share homology to known phages and plasmids. Taking all of these observations together, it is speculated that this unique genomic feature is the result of an off-target spacer integration event following the divergence of this strain lineage from the rest of the S. islandicus species.

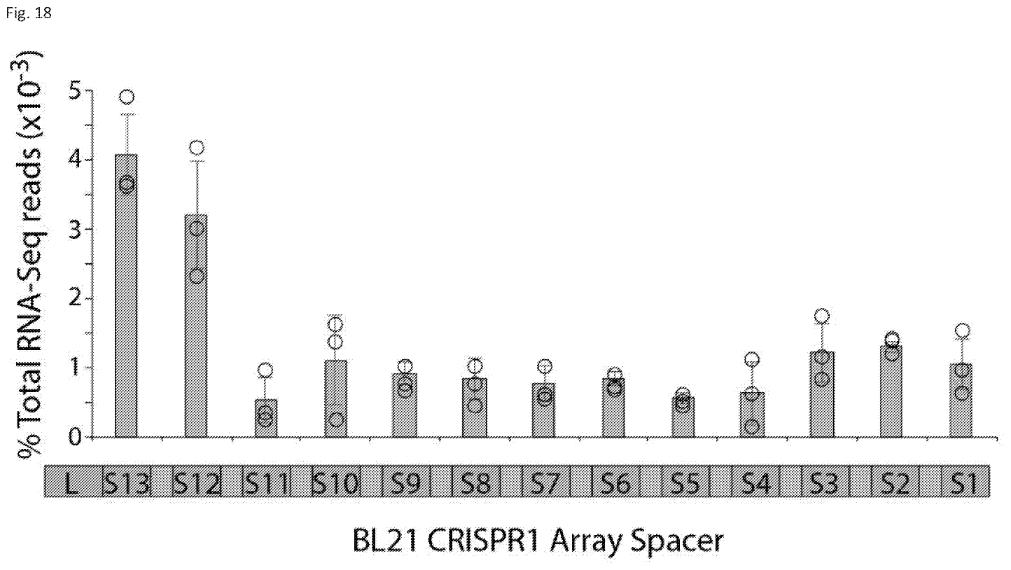

Spacer integration into the leader-proximal end of CRISPR loci is an essential phenomenon of CRISPR-Cas systems. However, whether spacer integrations occur outside of canonical CRISPRs, and the potential biological consequences of this were both previously unknown. The present disclosure surprisingly found that by methods using DSA, whole-genome sequencing, and Spacer-seq, that off-target spacer integrations can occur at many unique sites throughout the E. coli genome and carried plasmids. Off-target spacer integrations are potentially deleterious events that could affect genome integrity (Wright, A V., Doudna, J A. Protecting genome integrity during CRISPR immune adaptation. Nat. Struct and Mol. Biol. (10):876-883 (2016)). On the other hand, the process of evolution itself is predicated on chance sampling of beneficial mutations through lapses in genetic fidelity. It has previously been shown that spacer acquisition is optimized for integration into the leader proximal end of the array to achieve a robust immune response, as spacers at the trailing end of the array are poorly expressed (McGinn, J., and Marraffini, L. A. CRISPR-Cas systems optimize their immune response by specifying the site of spacer integration. Mol Cell 16, 30517-2 (2016)). This was found to be true in the case of E. coli BL21 CRISPR1 array expression as well (FIG. 18). Thus, off-target spacer integration activity, while likely deleterious in most instances, has the potential to boost crRNA expression levels and increase spacer diversity. To extend the relevance of our findings beyond our experimental model system, we also uncovered several examples of putative off-target spacer integration activity in previously sequenced genomes within the Y. pestis and S. islandicus lineages, and term this phenomenon “neo-CRISPR genesis.” As the number of whole-genome sequenced microbial species increases, particularly within clades of closely related strains, the present disclosure paves way for discovering further instances of neo-CRISPR genesis.

RNA Spacer-Seq and Analysis:

The total RNA content of the cell pellets were extracted and purified with a RNeasy Mini Kit (Qiagen) according to the manufacturer's protocol for bacterial cultures. The purified RNA was then used to produce cDNA using the ProtoScript II First Strand cDNA Synthesis Kit (NEB), and next made double-stranded with the Second Strand cDNA Synthesis protocol according to NEB. The double stranded cDNA was finally sheared, adaptor-ligated and subjected to the same protocol as the genomic DNA Spacer-seq process and analysis. To compare RNA Spacer-seq reads to total transcript abundance, a traditional RNA-Seq was also performed on the isolated total RNA.

### Plasmid Interference Assay:

The Neo-CRISPR arrays containing the M13 spacer were synthesized as gBlocks (IDT) and cloned by Gibson assembly into the pJKR-H-tetR vector (replacing the GFP gene downstream of the pLtetO promoter). Sequence-verified plasmids were transformed into E. coli K12 BW40114. A plasmid containing the M13 spacer target site was constructed by cloning the 33 bp target sequence into the pFN19K plasmid via PCR. A plasmid interference assay has been previously described (Datsenko, K. A., Pougach, K., Tikhonov. A., Wanner, B. L., Severinov. K., and Semenova, E. Molecular memory of prior infections activates the CRISPR/Cas adaptive bacterial immunity system. Nat. Commun. 3, 945. (2012)). Briefly, overnight cultures of strains containing the NCA plasmids were started from plates. In the morning, cultures were diluted into fresh LB containing the inducers arabinose, IPTG, and anhydrotetracycline (aTc; Clontech), and grown for an additional two hours. Cells were then washed 3× in cold water, transformed with 50 ng pFN19K+M13 target plasmid, and allowed to recover ˜1 hr in LB at 37 degrees before plating on LB+Kan plates (absolute efficiency) and LB+Carb plates (to normalize efficiencies).

Strain Knockouts:

BL21-AI strains containing IHF alpha and beta knockouts were a generous gift of J. Doudna (UC). The BL21-AI CRISPR1 array knockout strain and the OTCR strain were constructed by following the lambda-Red+Cas9 gene editing strategy (Jiang, Y., et al. Multigene editing in Escherichia coli genome via the CRISPR-Cas9 system. Appl. And Environment. Microbiol. 81(7), 2506-2514. (2015)).

Data Availability:

Spacer-Seq Illumina sequencing data has been deposited to NCBI SRA (BioSample accession SAMN08134321).

### Example V

### Embodiments

The following embodiments are contemplated by the present disclosure. A method of altering a cell is provided including the providing the cell with one or more nucleic acid sequences encoding a Cas1 protein and/or a Cas2 protein of a CRISPR adaptation system, providing the cell with a consensus CRISPR array nucleic acid sequence including a leader sequence and at least one repeat sequence which is a consensus sequence of a plurality of repeat sequences within off-target integration sites, wherein the consensus CRISPR array nucleic acid sequence is within genomic DNA of the cell or on a plasmid, and wherein the cell expresses the Cas1 protein and/or the Cas2 protein. According to one aspect, the cell is provided with one or more or a plurality of protospacer DNA sequences, and wherein the one or more or a plurality of protospacer DNA sequences is processed and a spacer sequence is inserted into the consensus CRISPR array nucleic acid sequence. According to one aspect, the protospacer sequence includes a modified “AAG” protospacer adjacent motif (PAM). According to one aspect, the one or more or plurality of protospacer sequences is a natural DNA sequence or a synthetic DNA sequence. According to one aspect, the nucleic acid sequence encoding the Cas1 protein and/or a Cas2 protein is provided to the cell within a vector or within one or more vectors. According to one aspect, the cell is a prokaryotic or a eukaryotic cell. According to one aspect, the nucleic acid sequence encoding the Cas1 protein and/or a Cas2 protein includes inducible promoters for induction of expression of the Cas1 and/or Cas2 protein. According to one aspect, the consensus repeat sequence is derived from a plurality of off-target integration site sequences. According to one aspect, the consensus repeat sequence is (5′)NNNNNCCNCGCGCGCGCGNGGNNNNNNN(3′) (SEQ ID NO: 1).

The disclosure provides an engineered, non-naturally occurring cell including one or more nucleic acid sequences encoding a Cas1 protein and/or a Cas2 protein of a CRISPR adaptation system, a consensus CRISPR array nucleic acid sequence including a leader sequence and at least one consensus repeat sequence, and wherein the consensus CRISPR array nucleic acid sequence is within genomic DNA of the cell or on a plasmid, and wherein the cell expresses the Cas1 protein and/or the Cas2 protein. According to one aspect, the engineered, non-naturally occurring cell further includes one or more or a plurality of protospacer sequences within the cell. According to one aspect, the engineered, non-naturally occurring cell includes at least one spacer sequence inserted into the consensus CRISPR array nucleic acid sequence, which spacer sequence was derived from a corresponding protospacer sequence exogenously provided to the cell. According to one aspect, the protospacer sequence includes a modified “AAG” protospacer adjacent motif (PAM). According to one aspect, the one or more or plurality of protospacer sequences is a natural DNA sequence or a synthetic DNA sequence. According to one aspect, the nucleic acid sequence encoding the Cas1 protein and/or a Cas2 protein is provided to the cell within a vector or within one or more vectors. According to one aspect, the cell is a prokaryotic or a eukaryotic cell. According to one aspect, the nucleic acid sequence encoding the Cas1 protein and/or a Cas2 protein comprises inducible promoters for induction of expression of the Cas1 and/or Cas2 protein. According to one aspect, the consensus repeat sequence is derived from a plurality of off-target integration site sequences. According to one aspect, the consensus repeat sequence is (5′)NNNNNCCNCGCGCGCGCGNGGNNNNNNN(3′) (SEQ ID NO: 1).

The disclosure provides a method of inserting a target DNA sequence within genomic DNA of a cell including providing the target DNA sequence to the cell, wherein the cell includes a nucleic acid sequence encoding a Cas1 protein and/or a Cas2 protein of a CRISPR adaptation system and a consensus CRISPR array nucleic acid sequence including a leader sequence and at least one consensus repeat sequence, wherein the cell expresses the Cas1 protein and/or the Cas2 protein and wherein the consensus CRISPR array nucleic acid sequence is within genomic DNA of the cell or on a plasmid, and wherein the Cas1 protein and/or the Cas2 protein processes the target DNA sequence and the target DNA sequence is inserted into the consensus CRISPR array nucleic acid sequence adjacent a corresponding consensus repeat sequence. According to one aspect, the target DNA sequence is a protospacer sequence including a modified “AAG” protospacer adjacent motif (PAM). According to one aspect, the target DNA sequence is a natural DNA sequence or a synthetic DNA sequence. According to one aspect, the nucleic acid sequence encoding the Cas protein and/or a Cas2 protein is provided to the cell within a vector or within one or more vectors. According to one aspect, the cell is a prokaryotic or a eukaryotic cell. According to one aspect, the nucleic acid sequence encoding the Cas1 protein and/or a Cas2 protein includes inducible promoters for induction of expression of the Cas1 and/or Cas2 protein. According to one aspect, the consensus repeat sequence is derived from a plurality of off-target integration site sequences. According to one aspect, the consensus repeat sequence is (5′)NNNNNCCNCGCGCGCGCGNGGNNNNNNN(3′) (SEQ ID NO: 1). According to one aspect, the step of providing is repeated such that a plurality of target DNA sequences are inserted into the consensus CRISPR array nucleic acid sequence at corresponding consensus repeat sequences.

The disclosure provides a nucleic acid storage system including an engineered, non-naturally occurring cell including one or more nucleic acid sequences encoding a Cas1 protein and/or a Cas2 protein of a CRISPR adaptation system, a consensus CRISPR array nucleic acid sequence including a leader sequence and at least one consensus repeat sequence, and wherein the consensus CRISPR array nucleic acid sequence is within genomic DNA of the cell or on a plasmid, and wherein the cell expresses the Cas1 protein and/or the Cas2 protein. According to one aspect, the consensus repeat sequence is derived from a plurality of off-target integration site sequences. According to one aspect, the consensus repeat sequence is (5′)NNNNNCCNCGCGCGCGCGNGGNNNNNNN(3′) (SEQ ID NO: 1). According to one aspect, at least one protospacer DNA sequence is provided to the cell and is processed and a spacer sequence is inserted into the consensus CRISPR array nucleic acid sequence.

The disclosure provides a system for in vivo molecular recording including an engineered, non-naturally occurring cell including one or more nucleic acid sequences encoding a Cas1 protein and/or a Cas2 protein of a CRISPR adaptation system, a consensus CRISPR array nucleic acid sequence including a leader sequence and at least one consensus repeat sequence, and wherein the consensus CRISPR array nucleic acid sequence is within genomic DNA of the cell or on a plasmid, and wherein the cell expresses the Cas1 protein and/or the Cas2 protein. According to one aspect, the consensus repeat sequence is derived from a plurality of off-target integration site sequences. According to one aspect, the consensus repeat sequence is (5′)NNNNNCCNCGCGCGCGCGNGGNNNNNNN(3′) (SEQ ID NO: 1).

The disclosure provides a kit for in vivo molecular recording including an engineered, non-naturally occurring cell including one or more nucleic acid sequences encoding a Cas1 protein and/or a Cas2 protein of a CRISPR adaptation system, a consensus CRISPR array nucleic acid sequence including a leader sequence and at least one consensus repeat sequence wherein the CRISPR array nucleic acid sequence is within genomic DNA of the cell or on a plasmid, one or more or a plurality of protospacer DNA sequences to be processed and introduced into the consensus CRISPR array, and optional instructions for use. The various components may be in separate containers or one or more components may be in the same container. According to one aspect, the consensus repeat sequence is derived from a plurality of off-target integration site sequences. According to one aspect, the consensus repeat sequence is (5′)NNNNNCCNCGCGCGCGCGNGGNNNNNNN(3′) (SEQ ID NO: 1).

What is claimed is:

1. A method of altering a cell comprising
providing the cell with one or more nucleic acid sequences encoding a Cas1 protein and/or a Cas2 protein of a CRISPR adaptation system,
providing the cell with a consensus CRISPR array nucleic acid sequence including a leader sequence and at least one repeat sequence which is a consensus sequence of a plurality of repeat sequences within off-target integration sites, wherein the consensus CRISPR array nucleic acid sequence is within genomic DNA of the cell or on a plasmid, and
wherein the cell expresses the Cas1 protein and/or the Cas2 protein.

2. The method of claim 1 wherein the cell is provided with one or more or a plurality of protospacer DNA sequences, and
wherein the one or more or a plurality of protospacer DNA sequences is processed and a spacer sequence is inserted into the consensus CRISPR array nucleic acid sequence.

3. The method of claim 2 wherein the protospacer sequence includes a modified “AAG” protospacer adjacent motif (PAM).

4. The method of claim 2 wherein the one or more or plurality of protospacer sequences is a natural DNA sequence or a synthetic DNA sequence.

5. The method of claim 1 wherein the nucleic acid sequence encoding the Cas1 protein and/or a Cas2 protein is provided to the cell within a vector or within one or more vectors.

6. The method of claim 1 wherein the cell is a prokaryotic or a eukaryotic cell.

7. The method of claim 1 wherein the nucleic acid sequence encoding the Cas1 protein and/or a Cas2 protein comprises inducible promoters for induction of expression of the Cas1 and/or Cas2 protein.

8. The method of claim 1 wherein the consensus repeat sequence is derived from a plurality of off-target integration site sequences.

9. The method of claim 1 wherein the consensus repeat sequence is
(SEQ ID NO: 1)
(5′)NNNNNCCNCGCGCGCGCGNGGNNNNNNN(3′).

10. An engineered, non-naturally occurring cell comprising
one or more nucleic acid sequences encoding a Cas1 protein and/or a Cas2 protein of a CRISPR adaptation system,
a consensus CRISPR array nucleic acid sequence including a leader sequence and at least one consensus repeat sequence, and
wherein the consensus CRISPR array nucleic acid sequence is within genomic DNA of the cell or on a plasmid, and
wherein the cell expresses the Cas1 protein and/or the Cas2 protein.

11. The engineered, non-naturally occurring cell of claim 10 further comprising one or more or a plurality of protospacer sequences within the cell.

12. The engineered, non-naturally occurring cell of claim 10 including at least one spacer sequence inserted into the consensus CRISPR array nucleic acid sequence, which spacer sequence was derived from a corresponding protospacer sequence exogenously provided to the cell.

13. The engineered, non-naturally occurring cell of claim 12 wherein the protospacer sequence includes a modified “AAG” protospacer adjacent motif (PAM).

14. The engineered, non-naturally occurring cell of claim 11 wherein the one or more or plurality of protospacer sequences is a natural DNA sequence or a synthetic DNA sequence.

15. The engineered, non-naturally occurring cell of claim 10 wherein the nucleic acid sequence encoding the Cas1 protein and/or a Cas2 protein is provided to the cell within a vector or within one or more vectors.

16. The engineered, non-naturally occurring cell of claim 10 wherein the cell is a prokaryotic or a eukaryotic cell.

17. The engineered, non-naturally occurring cell of claim 10 wherein the nucleic acid sequence encoding the Cas1 protein and/or a Cas2 protein comprises inducible promoters for induction of expression of the Cas1 and/or Cas2 protein.

18. The engineered, non-naturally occurring cell of claim 10 wherein the consensus repeat sequence is derived from a plurality of off-target integration site sequences.

19. The engineered, non-naturally occurring cell of claim 10 wherein the consensus repeat sequence is (5′)NNNNNCCNCGCGCGCGCGNGGNNNNNNN(3′) (SEQ ID NO: 1).

20. A method of inserting a target DNA sequence within genomic DNA of a cell comprising
providing the target DNA sequence to the cell, wherein the cell includes a nucleic acid sequence encoding a Cas1 protein and/or a Cas2 protein of a CRISPR adaptation system and a consensus CRISPR array nucleic acid sequence including a leader sequence and at least one consensus repeat sequence,
wherein the cell expresses the Cas1 protein and/or the Cas2 protein and wherein the consensus CRISPR array nucleic acid sequence is within genomic DNA of the cell or on a plasmid, and
wherein the Cas1 protein and/or the Cas2 protein processes the target DNA sequence and the target DNA sequence is inserted into the consensus CRISPR array nucleic acid sequence adjacent a corresponding consensus repeat sequence.

21. The method of claim 20 wherein the target DNA sequence is a protospacer sequence including a modified “AAG” protospacer adjacent motif (PAM).

22. The method of claim 20 wherein the target DNA sequence is a natural DNA sequence or a synthetic DNA sequence.

23. The method of claim 20 wherein the nucleic acid sequence encoding the Cas1 protein and/or a Cas2 protein is provided to the cell within a vector or within one or more vectors.

24. The method of claim 20 wherein the cell is a prokaryotic or a eukaryotic cell.

25. The method of claim 20 wherein the nucleic acid sequence encoding the Cas1 protein and/or a Cas2 protein comprises inducible promoters for induction of expression of the Cas1 and/or Cas2 protein.

26. The method of claim 20 wherein the consensus repeat sequence is derived from a plurality of off-target integration site sequences.

27. The method of claim 20 wherein the consensus repeat sequence is (5′)NNNNNCCNCGCGCGCGCGNGGNNNNNNN(3′) (SEQ ID NO: 1).

28. The method of claim 20 wherein the step of providing is repeated such that a plurality of target DNA sequences are inserted into the consensus CRISPR array nucleic acid sequence at corresponding consensus repeat sequences.

29. A nucleic acid storage system comprising
an engineered, non-naturally occurring cell including
one or more nucleic acid sequences encoding a Cas1 protein and/or a Cas2 protein of a CRISPR adaptation system,
a consensus CRISPR array nucleic acid sequence including a leader sequence and at least one consensus repeat sequence, and
wherein the consensus CRISPR array nucleic acid sequence is within genomic DNA of the cell or on a plasmid, and
wherein the cell expresses the Cas1 protein and/or the Cas2 protein.

30. The nucleic acid storage system of claim 29 wherein the consensus repeat sequence is derived from a plurality of off-target integration site sequences.

31. The nucleic acid storage system of claim 29 wherein the consensus repeat sequence is
(SEQ ID NO: 1)
(5′)NNNNNCCNCGCGCGCGCGNGGNNNNNNN(3′).

32. The nucleic acid storage system of claim 29 wherein at least one protospacer DNA sequence is provided to the cell and is processed and a spacer sequence is inserted into the consensus CRISPR array nucleic acid sequence.

33. A system for in vivo molecular recording comprising
an engineered, non-naturally occurring cell including
one or more nucleic acid sequences encoding a Cas1 protein and/or a Cas2 protein of a CRISPR adaptation system,
a consensus CRISPR array nucleic acid sequence including a leader sequence and at least one consensus repeat sequence, and
wherein the consensus CRISPR array nucleic acid sequence is within genomic DNA of the cell or on a plasmid, and
wherein the cell expresses the Cas1 protein and/or the Cas2 protein.

34. The system for in vivo molecular recording of claim 33 wherein the consensus repeat sequence is derived from a plurality of off-target integration site sequences.

35. The system of claim 33 wherein the consensus repeat sequence is
(SEQ ID NO: 1)
(5′)NNNNNCCNCGCGCGCGCGNGGNNNNNNN(3′).

36. A kit for in vivo molecular recording comprising
an engineered, non-naturally occurring cell including
one or more nucleic acid sequences encoding a Cas1 protein and/or a Cas2 protein of a CRISPR adaptation system,
a consensus CRISPR array nucleic acid sequence including a leader sequence and at least one consensus repeat sequence wherein the CRISPR array nucleic acid sequence is within genomic DNA of the cell or on a plasmid,
one or more or a plurality of protospacer DNA sequences to be processed and introduced into the consensus CRISPR array, and
optional instructions for use.

37. The system for in vivo molecular recording of claim 36 wherein the consensus repeat sequence is derived from a plurality of off-target integration site sequences.

38. The system of claim 36 wherein the consensus repeat sequence is
(SEQ ID NO: 1)
(5′)NNNNNCCNCGCGCGCGCGNGGNNNNNNN(3′).
